(19) United States
(12) Patent Application Publication
(10) Pub. No.: US 2026/0307085 A1
(43) Pub. Date: Oct. 8, 2026

(54) NOZZLE MANIFOLD ASSEMBLY

(71) Applicant: The Aerospace Corporation, El Segundo, CA (US)

(72) Inventor: James M. HELT, Playa del Rey, CA (US)

(73) Assignee: The Aerospace Corporation, El Segundo, CA (US)

(21) Appl. No.: 19/711,766

(22) Filed: Jun. 17, 2026

Related U.S. Application Data
(63) Continuation-in-part of application No. 19/302,285, filed on Aug. 18, 2025.
Application No. 19/302,285 is a continuation-in-part of application No. 18/986,367, filed on Dec. 18, 2024.

(51) Int. Cl. B05B 1/02; G01N 21/84; G01N 33/30

(52) U.S. Cl. CPC B05B 1/02; G01N 21/8422; G01N 33/30

(57) ABSTRACT

A nozzle manifold assembly (NMA) for a reflective lens (RFL) includes a nozzle manifold (“manifold”) and a nozzle being part of the NMA. For example, the NMA and the RFL may be used in constructing fluid-surface inspection and analysis instruments like RFL based thin film viscometers (RFL-TFVs). The manifold is configured to accept a fluid supply to form a jet, and configured to include and or mount to the nozzle. The manifold may include a plurality of sensor-control devices configured to measure and/or control one or more properties of the NMA, the RFL, and/or the jet. The NMA also includes a gas supply connected to, and supplying fluid (gas, liquid etc.) through, the manifold to form the jet.

### CROSS REFERENCE TO RELATED APPLICATIONS

This application claims priority to, and is a continuation-in-part of, U.S. application Ser. No. 19/302,285, filed on Aug. 18, 2025, which claims priority to, and is a continuation-in-part of, U.S. application Ser. No. 18/986,367, filed on Dec. 18, 2024. The subject matter of the related applications is incorporated herein by reference.

### FIELD

The present invention relates to an imaging lens for inspection and analysis, and more particularly, to a nozzle manifold assembly (NMA) for reflective lens or reflective imaging lens (RFL).

### BACKGROUND

National Security Space (NSS) utilizes specialty fluids in a range of applications where they serve to lubricate tribological contacts, as coatings precursors, as heat transfer media, as dampening and hydraulic fluids. For instance, when a spacecraft moving mechanical assembly (MMA) is placed in space, including a control moment gyroscope (CMG) or reaction wheel assembly (RWA), there is often a finite supply of lubricant.

Due to the finite lubricant supply, it is important to understand how the quantity and physical properties of the MMA's lubricant evolves over time during operation in space. This process of evolution includes understanding changes in the lubricant's viscosity due to tribological degradation, which directly impacts how much lubricant life may be left for optimal performance.

It should also be noted that it is important to understand why a lubricant's local viscosity is critical to tribology. For example, the change in the lubricant's viscosity with use correlates to the lubricant's health, the remaining “life” of the lubricant, and the counterbody tribological performance. In one example, a lubricant's viscosity is a key physical property found in elastohydrodynamic lubrication (EHL) theory that is used to model and predict attitude control mechanism bearing performance. Changes to a lubricant's viscosity impact EHL film thickness and entrainment dynamics calculations.

Accordingly, there is a need to develop a technique to measure space-based liquid (e.g., fluid lubricant) physical property evolution.

### SUMMARY

Certain embodiments of the present invention may provide solutions to the problems and needs in the art that have not yet been fully identified, appreciated, or solved by current liquid evolution measurement technologies. For example, some embodiments of the present invention pertain to a nozzle manifold assembly (NMA) for reflective lens or reflective imaging lens (hereinafter “RFL”). In some embodiments, the NMA and the RFL are two separate components that are combined to form the RFL NMA device. In another embodiment, the RFL NMA device is designed and constructed as a single component that includes NMA and RFL features needed for a particular inspection-analysis application. In an embodiment, the NMA device is used to construct the RFL based thin film viscometers (RFL-TFVs), which can measure the interfacial-rheological-optical properties of thin fluid films and the surfaces that they coat.

In one embodiment, a NMA for a RFL includes a nozzle manifold (“manifold”) and a nozzle being part of the NMA. The manifold is configured to accept fluid, gas and/or liquid, supply to form a jet, and configured to mount to the nozzle. The manifold may include a plurality of sensors configured to measure and/or control one or more properties of the NMA, the RFL, and/or the jet. The NMA also includes a fluid, gas and/or liquid, supply connected to, and supplying fluid through, the manifold to form the jet.

### BRIEF DESCRIPTION OF THE DRAWINGS

In order that the advantages of certain embodiments of the invention will be readily understood, a more particular description of the invention briefly described above will be rendered by reference to specific embodiments that are illustrated in the appended drawings. While it should be understood that these drawings depict only typical embodiments of the invention and are not therefore to be considered to be limiting its scope, the invention will be described and explained with additional specificity and detail through the use of the accompanying drawings, in which:

FIGS. 1A and 1B are generalized diagrams illustrating a close-up and plan (top) view of the RFL NMA's nozzle and the relative geometric arrangement to a substrate coated with a thin fluid film, according to an embodiment of the present invention.

FIG. 1C is a diagram illustrating an alternative nozzle configuration, which is part of the RFL NMA, according to an embodiment of the present invention.

FIGS. 2A-F are diagrams illustrating various views of a spider vane based RFL NMA, according to an embodiment of the present invention.

FIGS. 3A-F are diagrams illustrating various views of a RFL NMA incorporating optically transparent windows (or lenses), according to an embodiment of the present invention.

FIGS. 4A-D are diagrams illustrating various views of an alternative RFL NMA incorporating spider vanes and at least one optically transparent window (or lens), according to an embodiment of the present invention.

FIGS. 5A-C are diagrams illustrating a configuration for triangulating the RFL NMA's position with a position sensitive detector (PSD), according to an embodiment of the present invention.

FIG. 6 is a diagram illustrating a camera triangulation schematic of a RFL NMA, according to an embodiment of the present invention.

FIGS. 7A and 7B show images illustrating the use of camera triangulation, according to an embodiment of the present invention.

FIGS. 8A-D are diagrams illustrating various views of a RFL NMA with a modular heat-cooling device for temperature control, according to an embodiment of the present invention.

FIG. 9A-F are diagrams illustrating use of a modular RFL NMA configuration with a portable imaging device (e.g., smartphone), with two separate mounts and stands for independent vertical and angular positioning of the RFL, smartphone and NMA, according to an embodiment of the present invention.

### DETAILED DESCRIPTION OF THE EMBODIMENTS

Some embodiments of the present invention pertain to a nozzle manifold assembly (NMA) for reflective imaging lens (RFL) that is intended to be used to construct RFL NMA based thin film viscometers (RFL-TFVs) capable of measuring the interfacial-rheological-optical properties of thin fluid films and the surfaces that they coat. In these embodiments, NMA includes multiple features and components that facilitate mounting, incorporating, and/or aligning the NMA to (or within) a RFL used with film thickness and optical analysis-inspection instruments. For these instruments the NMA facilitates alignment, preferably coaxial, of the film thickness measurement location and/or optical image with a normal impinging jet's stagnation point, and the simultaneous monitoring and control of the impinging jet, RFL and NMA components. NMA attributes are preferred for construction of RFL-TFV instruments as they enable routine, local physical property analysis of fluids resting on solid material surfaces relevant to space mechanisms, simplify the measurement process, expand measurement protocols, improve measurement accuracy and precision, and ease theoretical analysis.

NMAs may be configured or designed in various ways depending on the RFL's design, application needs, and geometric constraints imposed by the RFL optical system and the article under analysis. For example, the RFL NMA may be used in constructing a RFL-TFV with a custom or suitably sealed, off-the-shelf RFL like microscope objectives. Custom RFL NMAs include designs that incorporate the NMA features directly into RFL structural elements such as the housing or barrel. In another example, NMA devices can be mounted to off-the-shelf RFLs and therefore integrate directly with commercial film thickness instruments and/or infrared microscopes, enabling measurements of fluid-coated articles with a direct line of sight.

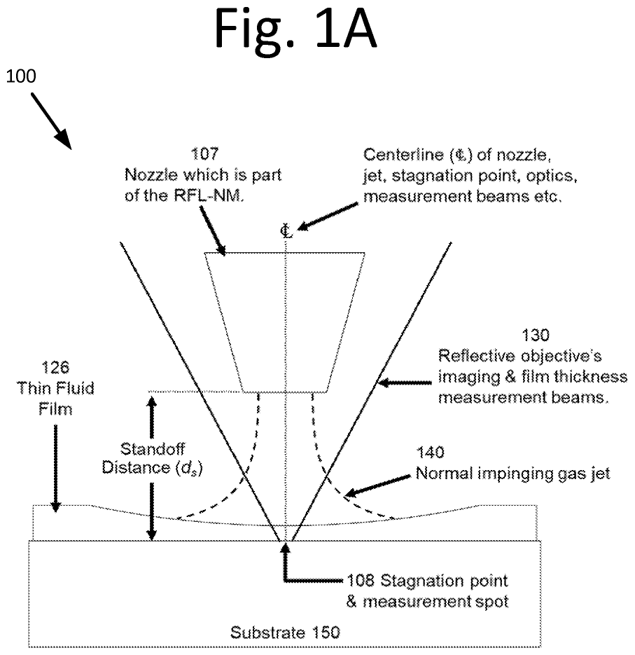

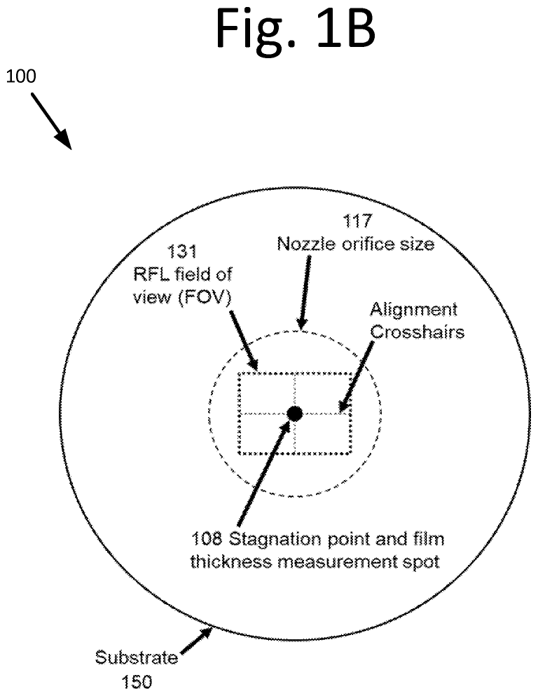

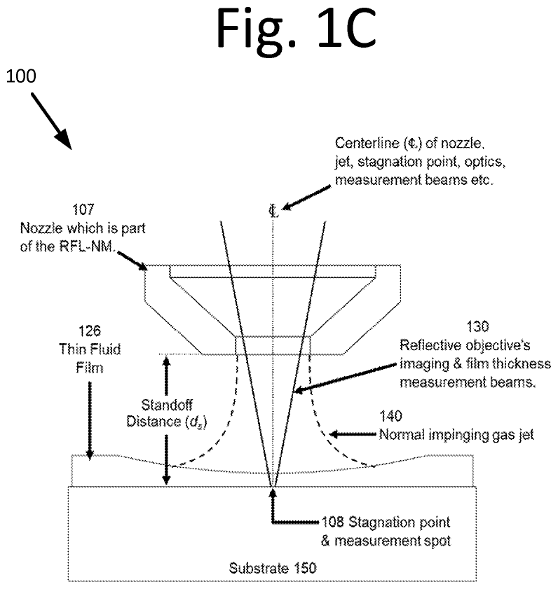

FIGS. 1A-C are diagrams illustrating an RFL NMA 100, according to an embodiment of the present invention. In some embodiments, RFL NMA 100 includes a nozzle 107 with a centerline of nozzle, jet, stagnation point (and film thickness measurement spot) 108, and optical and measurement beam(s). An imaging and film thickness measurement beam (“measurement beam”) 130 is transmitted down towards the substrate 150 along the centerline, and is configured to be aligned with the center of nozzle 107. Depending on the embodiment, the imaging beam and the film thickness measurement beam may be one in the same. RFL NMA 100 make measurements, with measurement beam 130, at the projected center of the nozzle 107 orifice onto substrate 150. In some embodiments, emitted out of nozzle 107 is an impinging gas (fluid) jet 140 that impinges orthogonal on surface of thin film fluid 126. Below thin film fluid 126, is a substrate 150. For purposes of explanation, the terms “gas jet”, “impinging gas jet” and “fluid jet” may be used interchangeably. Thin fluid film 126 may be reduced in thickness as gas jet 140 impinges thereon. It should be appreciated that, depending on the embodiment, any fluid which impinges on the surface of thin fluid film 126, may be used (e.g. gas, liquid, plasma etc.). Although not illustrated in this embodiment, mounted on or within the RFL NMA is a mirror, lens, window, nozzle 107, and sensors, to name a few. Additionally, and as depicted in FIGS. 1A-C, the nozzle 107, jet 140, jet stagnation point 108, optical and measurement beam 130 are in coaxial alignment, and therefore the centers of the jet stagnation point and measurement spot 108 are aligned.

In fluid dynamics, the location at which the fluid (or gas) jet 140 impinges on the surface (or coating) of thin fluid film 126 is called the stagnation point 108 and is a unique area of impinging gas jet 140. The theoretical background of stagnation point flow is described in literature and allows for the viscosity of the fluid to be measured from film thickness (h)−time (t) series data.

It should be appreciated that thin film viscometer (TFV) type instruments use stagnation flow models to measure a fluid's viscosity (η) and/or the jet's hydrodynamic constant (α). For Stokes flow, both parameters are contained in G which is termed the strength of the stagnation flow, as shown in Equation (1) below.

$$h ( t ) = \frac{{h}_{0}}{1 + {h}_{0} Gt} \qquad Equation ( 1 )$$

To construct and use TFV for viscosity measurements, a calibration curve of the jet's hydrodynamic constant (α) is determined using fluids of known viscosities. Next, measuring and fitting h(t) thinning profiles is performed as a function of relevant parameters including the type of gas used (e.g. nitrogen), gas properties, gas flow rate (Q), nozzle geometry and standoff distance (dₛ). Standoff distance (dₛ) is the distance between the substrate surface (i.e., not the surface of the thin fluid film) and the tip of the nozzle. Once calibrated, h(t) thinning profiles of unknown thin fluid films is measured, and the fluid's viscosity is calculated by data fitting.

As shown in FIG. 1B, the plan (top) view shows the locations of RFL's image field of view (FOV) 131, size of nozzle orifice 117, jet stagnation point and film thickness measurement spot 108 when coaxially aligned. It should be noted that the term “when coaxially aligned” is used, because there are various ways to measure the film's thickness when using the RFL NMA. For example, fiber optic spectral reflectance and ellipsometry make measurements at a specific spot like shown in the FIGS. 1A-C. In principle, one can move this measurement spot around in the image frame if they want. A person of ordinary skill in the art may do this if they're interested in making measurements outside the stagnation zone. It should be noted that the terms stagnation “zone” and “point” can be used interchangeably. Other film thickness techniques, such as interferometry, can determine the film's thickness anywhere in the image frame including the stagnation point which is preferably at the center of the image. Also shown in FIG. 1B is the alignment crosshairs, which are overlayed on digital video images obtained when using the RFL NMA with, for example, a microscope or other imaging system. These digital video images may be acquired and analyzed by a personal computer (PC). The size of nozzle orifice 117 in this embodiment may be larger than FOV 131, and part of the impingement area may be observed during the film thinning process. In such an embodiment, the stagnation point may be observed in the image of the RFL, allowing for measurements to be performed. Depending on the design and configuration, different types of nozzles 107 may be used. These nozzles may include orifices of varying sizes, number and arrangement. In those embodiments, the impingement area may be smaller than FOV 131.

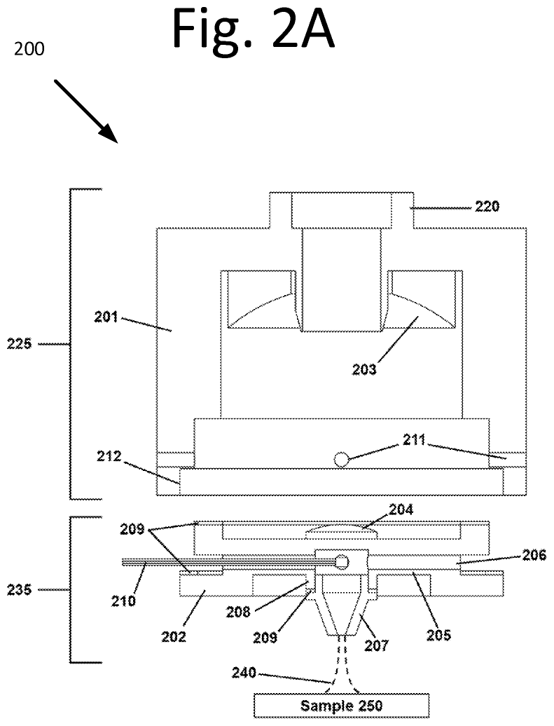

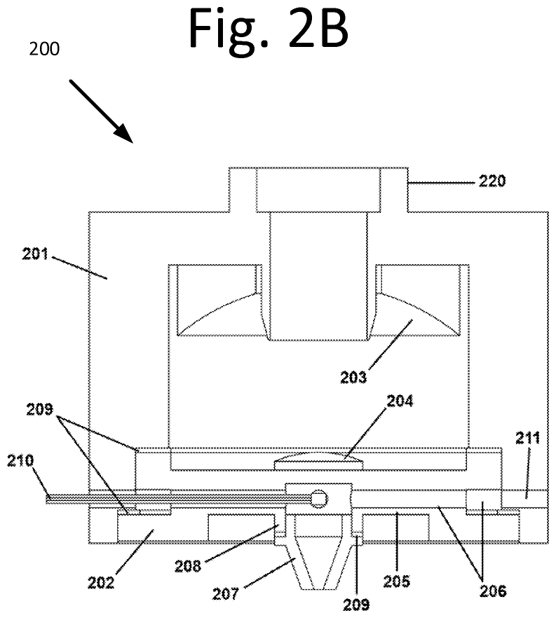

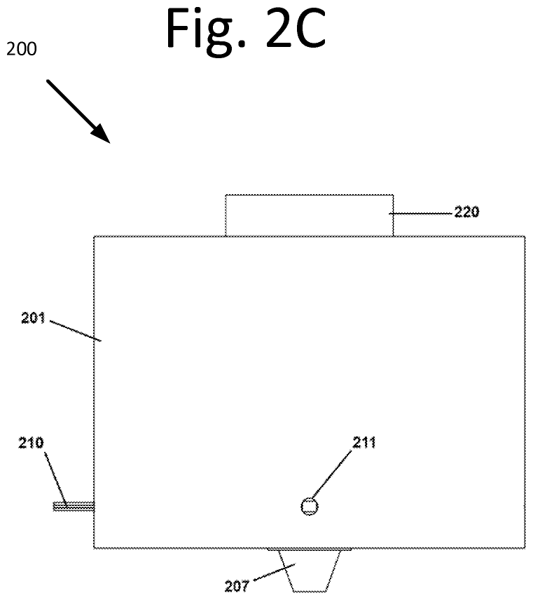

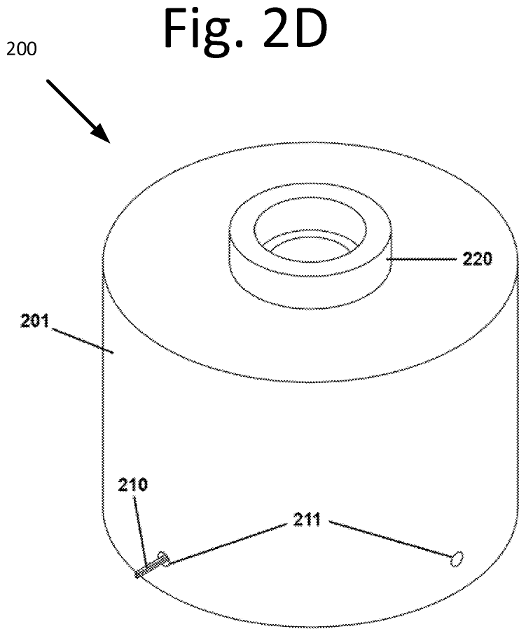

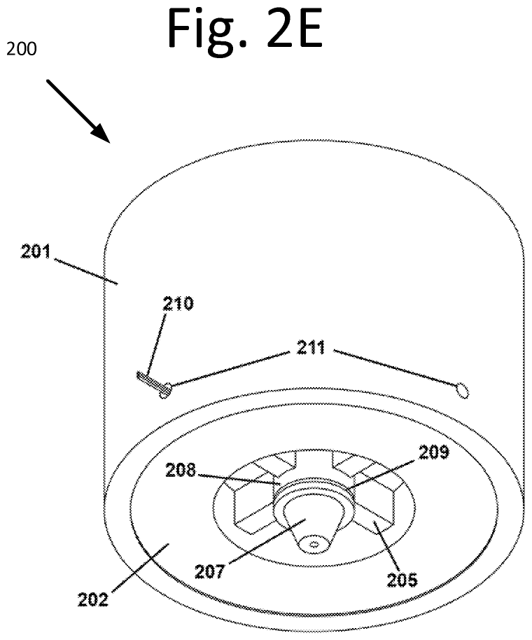

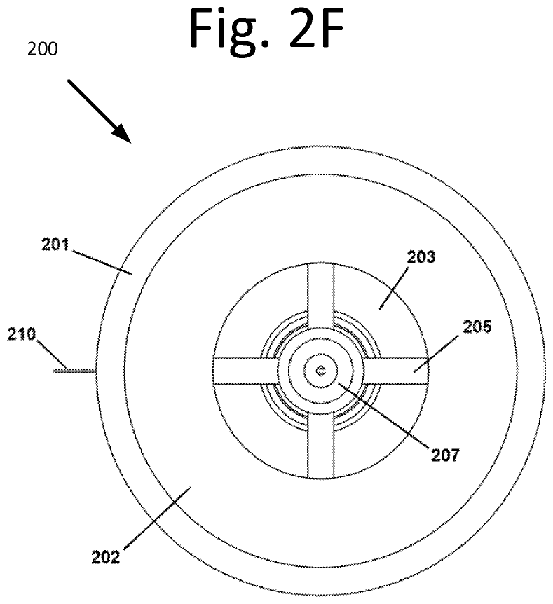

In other embodiments, such as that shown in FIG. 1C, an alternative nozzle 107 may be part of RFL NMA 100, according to an embodiment of the present invention. In this embodiment, measurement beams 130 are transmitted inside of nozzle 107, passing through the nozzles' orifice. This is different from measurement beams 130 of FIG. 1A that transmit outside of nozzle 107. As described in the embodiments of FIGS. 2-4, and 9, the choice between the different configurations illustrated in FIGS. 1A and 1C is a matter of engineering choice depending on the needs of the application. Elements guiding the configuration choice include spectral range and resolution, signal quality, application (e.g., characterization of fluid film, nozzle, solid film etc.), cost, and nozzle (NMA) interchangeability.

In some embodiments, the RFL uses reflective, mirrored optical elements, like those in reflective microscope objectives, that are mounted to or within structural body(s). In this instance, the RFL generally includes a lower body that mounts to the upper body, both of which possess reflective, mirrored optical elements for the imaging and measurement beams (130 in FIG. 1). In another embodiment, the RFL's reflective optics are mounted within a body that is intended to be coupled directly with digital imaging sensors like CCDs. In this embodiment, the RFL possesses mounting threads at one of the body's end. See, for example, FIGS. 2-4 and 9, which are diagrams illustrating various views of the RFL NMA 200, 300, 400 and 900, according to an embodiment of the present invention. Furthermore, RFL NMA 200, 300, 400, 800, and 900 described in the present invention use the general layout of a reverse Cassegrain-Schwarzschild reflective imaging objective as described in literature. It is understood the one or more of these embodiments may be used with other RLF designs, including those that are not symmetric.

In FIGS. 2A-F, the bodies of RFL NMA 200 are axially symmetric. Although RFL NMA 200 may be used with other reflective imaging lens designs, in these embodiments, RFL NMA 200 uses the general layout of a reverse Cassegrain-Schwarzschild reflective imaging objective as described in literature. It is understood the invention can be used with other RLF designs, including those that are not symmetric.

In some embodiments, such as that shown in FIGS. 2A-F, RFL NMA 200 uses a RFL upper assembly 225, which is comprised of upper body 201 with primary mirror 203 attached, and NMA 235. RFL NMA 200's nozzle manifold assembly 235 is comprised of lower body 202 that includes a spider mount possessing spider vanes 205 and secondary mirror 204 attached to the top-center of the spider mount. In these embodiments, the nozzle 207 of lower NMA 235 mounts to the bottom-center of the spider mount. It should be appreciated that, although nozzle 207 for NMAs are depicted removable, in certain embodiments, nozzle 207 may be permanent (i.e., integrated into the NMA as a single unit). In these embodiments, NMA 235 includes spider mount with spider vanes 205 possessing various channels, holes, bores, and features 206. When the RFL NMA 200 is assembled, features 206 and holes/ports 211 are used to integrate sensors (e.g. temperature and pressure) and supply fluid (e.g. gas) to nozzle 207 that forms the impinging jet during use.

As shown in FIG. 2A, RFL NMA 200 includes an upper assembly 225 and lower nozzle manifold assembly 235 in which lower body 202 mounts onto or into upper body 201 with features 212. Once these two assemblies, 225 and 235, are mounted together, the RFL NMA 200 is complete and may be incorporated into scientific instruments such as optical microscopes.

FIGS. 2B-F depict various views of the constructed RFL NMA 200. FIGS. 2B-C respectively depict cut-way and solid side-views illustrating RFL NMA 200 features accessible after construction, including ports/holes 206 and 211 used for sensor integration and to supply gas (fluid) for the impinging jet 240. Similarly, FIGS. 2D-F respectively depict solid-views with a top-perspective, bottom-perspective and bottom view illustrating the arrangement of the spider vanes 205 that incorporate channels/holes/bores to feed gas (fluid) to the nozzle 207.

Amongst other things assembled NMA 235, which mounts onto or into upper body 201 to form the RFL NMA, possesses features or components including lower body 202 with spider mount and spider vanes 205, features for gas supply 206 in (or on) the spider vanes, nozzle 207, nozzle mount 208, gaskets and/or seals 209, temperature sensor 210, features for integration (e.g. holes, bores, channels) and other sensors for control-monitoring NMA and jet properties (e.g. temperatures, nozzle pressure, barometric pressure, humidity etc.).

Depending on the embodiment, RFL NMA 200 may be constructed as a single unit, two separate units, or three or more separate units each composed of specific features and components (i.e. sub-assemblies). For a single unit construction, NMA 235 features and components are incorporated directly into the RFL's body and structure. For clarity RFL NMA 200, depicted in FIG. 2A, is comprised of two separate sub-assemblies (RFL upper assembly 225 with primary mirror 203, and lower NMA 235 with secondary mirror 204, spider mount/vanes 205 and nozzle 207). RFL NMA 200 may include the general components of a reflective imaging lens, including the housing, mirrors, reflective optics, stray light baffle, refractive optical elements for catadioptric (refractive-reflective) systems, upper body, primary mirror, lower body, spider mount/vanes, and secondary mirror to name a few.

The NMA 235 of the RFL NMA 200 includes features for mounting the NMA 235 to upper assembly 225 as depicted in FIGS. 2B-F. NMA 235's lower body 202 includes a plurality of channels/holes/bores 206 for the jet supply inlet, integrating sensors and temperature-pressure monitoring and/or control. RFL NMA 200 also includes a nozzle 207, gaskets (including seals, glue, etc.) 209, and a jet temperature sensor 210. In this embodiment, gaskets/seals 209 ensure the gas/liquid supplied to the RFL NMA 200 only exits through the nozzle 207 to create the impinging jet 240. FIG. 2A illustrates the use of a wired temperature sensor 210, like a thermocouple or platinum RTD, that is placed through one of a plurality of channels/holes/bores 206 in the lower body 202. While not shown, it is understood that temperature sensor 210 is sealed into hole 206 after mounting to prevent the jet's gas supply from leaking through hole 206. It should be noted that when constructing RFL NMA 200, it may be more convenient to insert temperature sensor 210 through port/holes 211 of upper body 201 and channels/holes/bores 206 in lower body 202 after mounting lower body 202 to upper body 201. As with other elements of the invention, this is a matter of design.

RFL NMA 200 can include other sensors, such as those to measure and/or control the jet's nozzle pressure, humidity and/or flow rate. In one embodiment, RFL NMA 200 includes pressure sensors (not shown) to measure barometric and/or nozzle differential (gage) pressure. Pressure sensors can, for example, be placed in one of a plurality of port/holes 211 of upper body 201 and/or channels/holes/bores 206 in the lower body 202. In another embodiment, RFL NMA 200 can include infrared sensors, high emissivity material for non-contact temperature sensing, pyrometers and cameras etc. (not shown) for measuring properties like the temperature of NMA components (jet 240, nozzle 207, lower body 202 etc.), atmosphere, and sample 250 being inspected and analyzed.

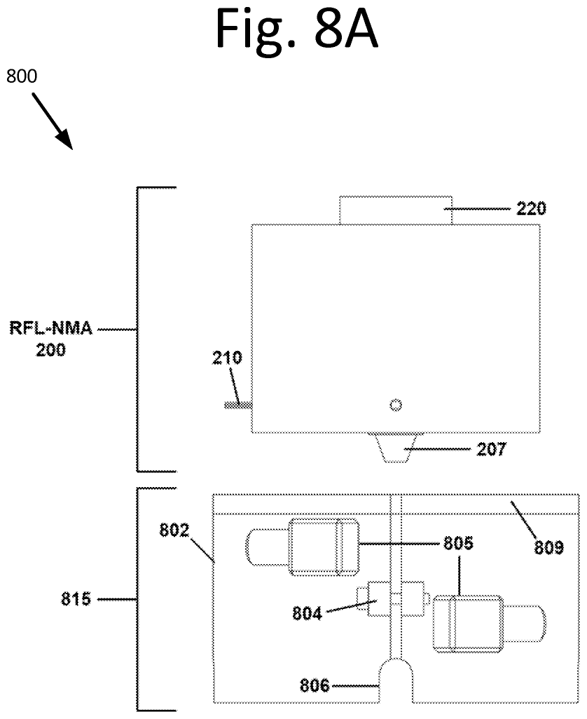

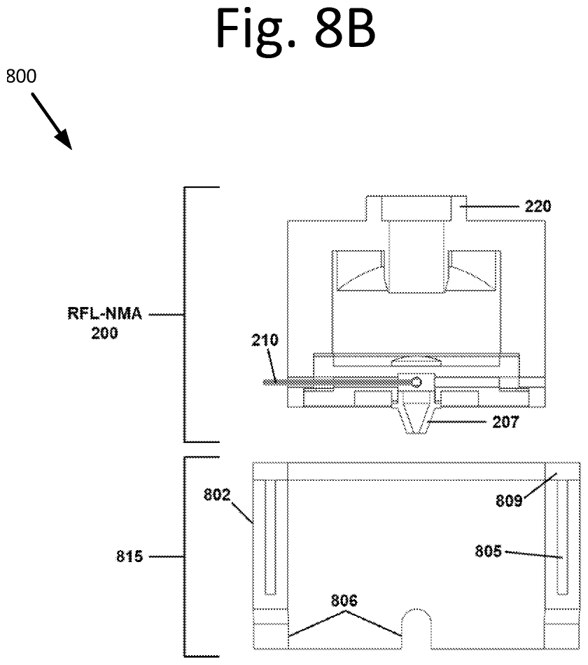

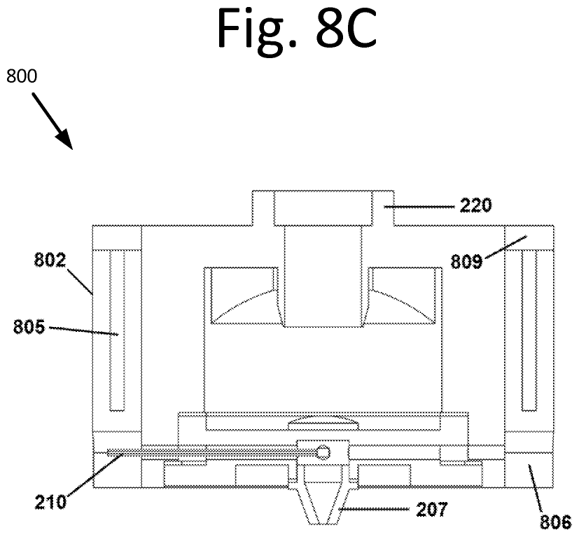

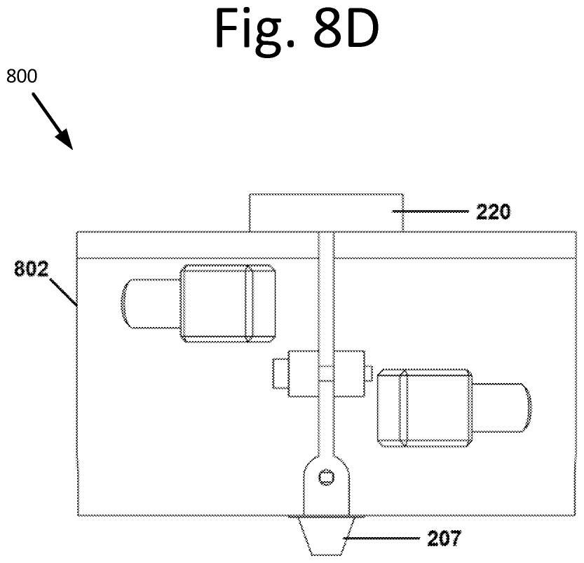

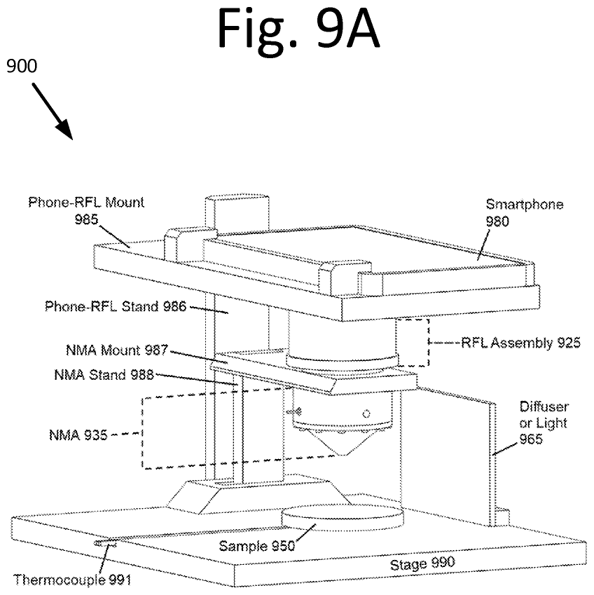

It is important to recognize that, for precise measurement of the impinging jet's temperature with either a wired sensor 210 or a pyrometer, one should consider thermal isolation of the sensor or high emissivity material when constructing the RFL NMA. In this embodiment, a high emissivity material is incorporated into the spider mount/vanes, nozzle or elsewhere for temperature sensing purposes. A pyrometer may then be used to measure the temperature of the high emissivity material, and therefore, the jet and/or other NMA components. It is important to recognize that precise measurement of the impinging jet's temperature with either a wired sensor or a pyrometer one should consider thermal isolation of the sensor or high emissivity material when constructing the RFL NMA. RFL NMA 200 further includes a RFL mount for the optical system 220, RFL imaging and film thickness measurement beams 230 (not shown), gas jet 240, and heating and cooling (not shown). The heating and cooling may be accomplished in various ways including use of a removable heating and cooling jacket like that shown in FIG. 8, using a surface mount heater/cooler like a Peltier device, integrating channels into the nozzle manifold body, or using an external heating and cooling of the jet supply.

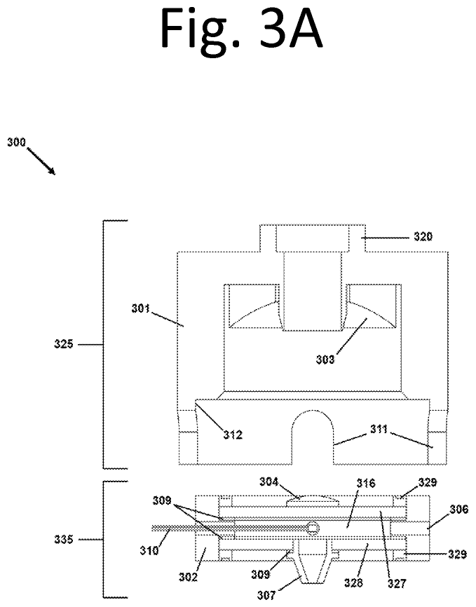

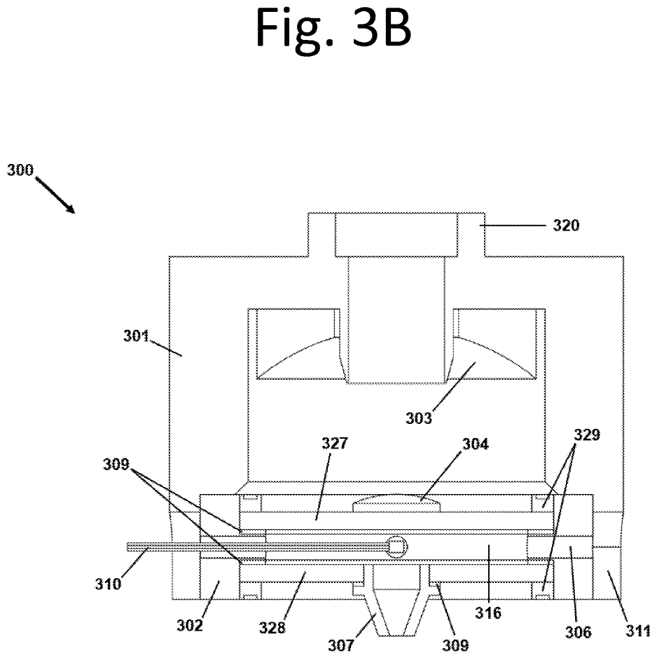

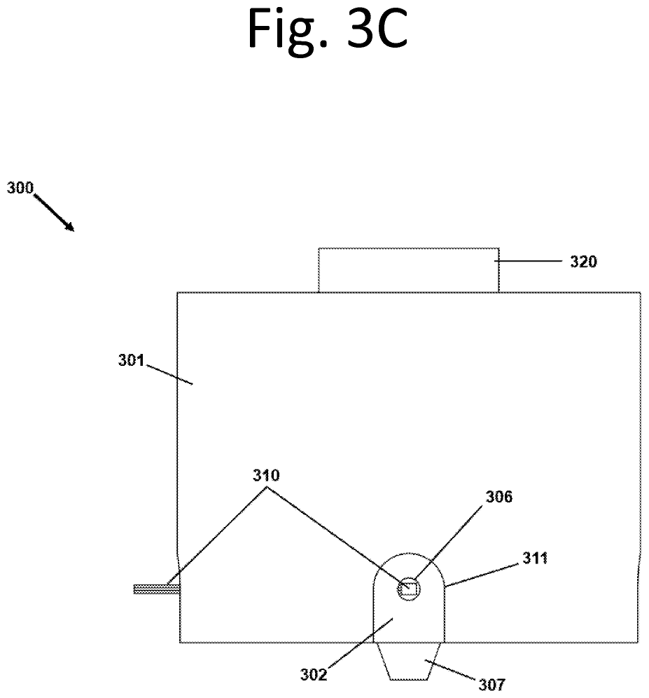

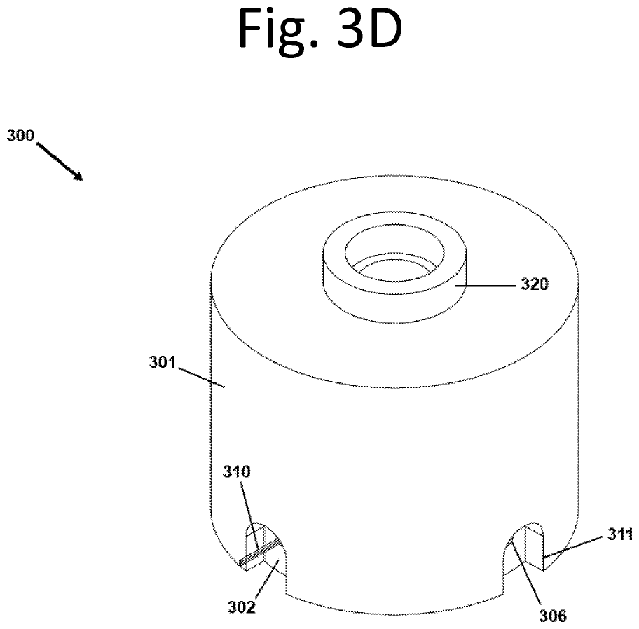

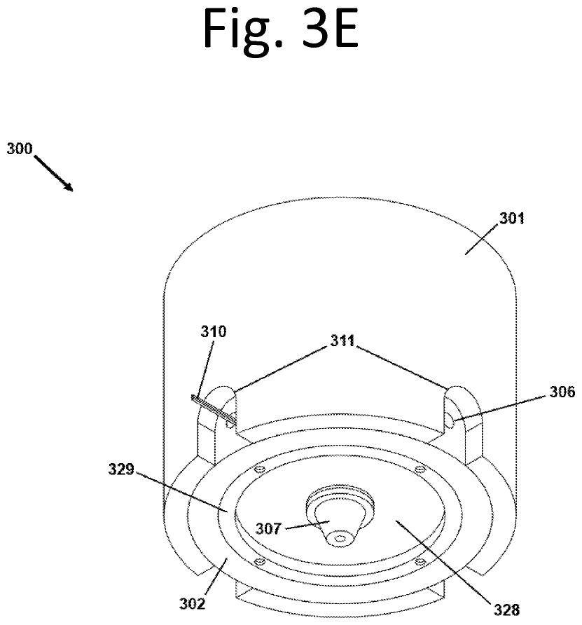

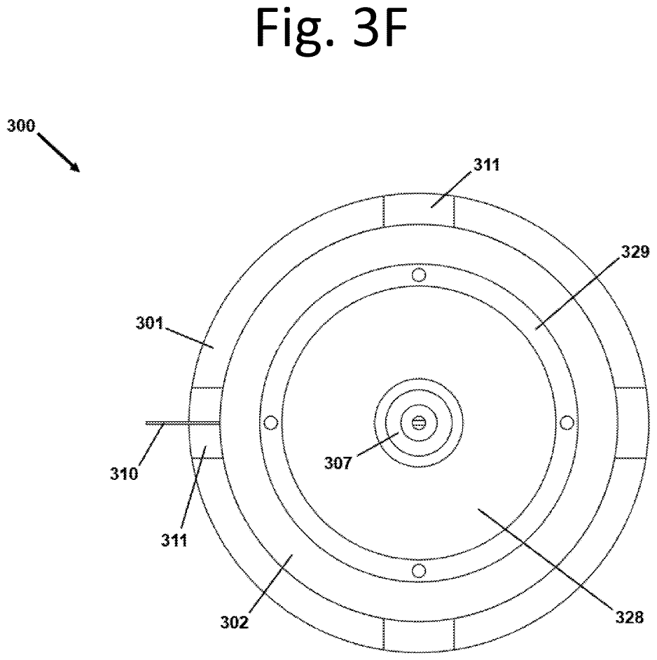

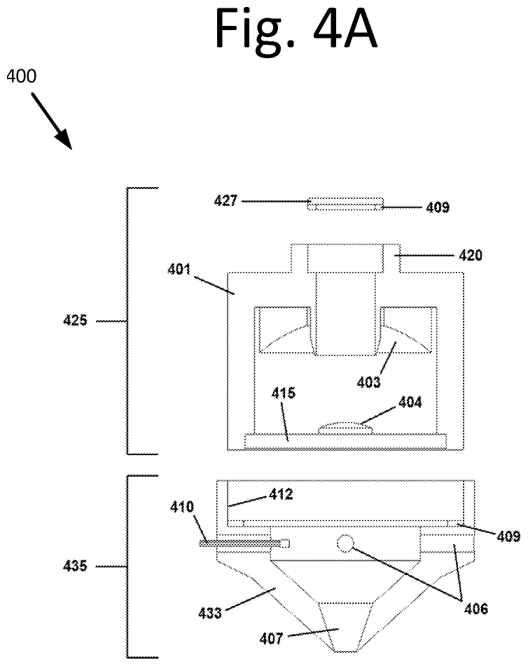

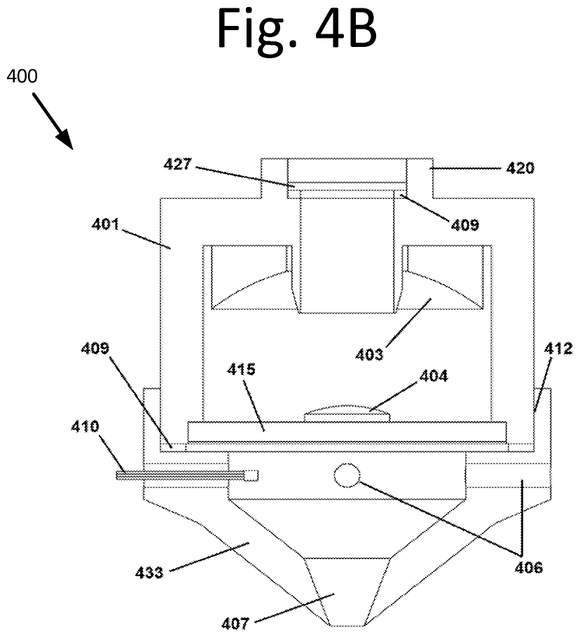

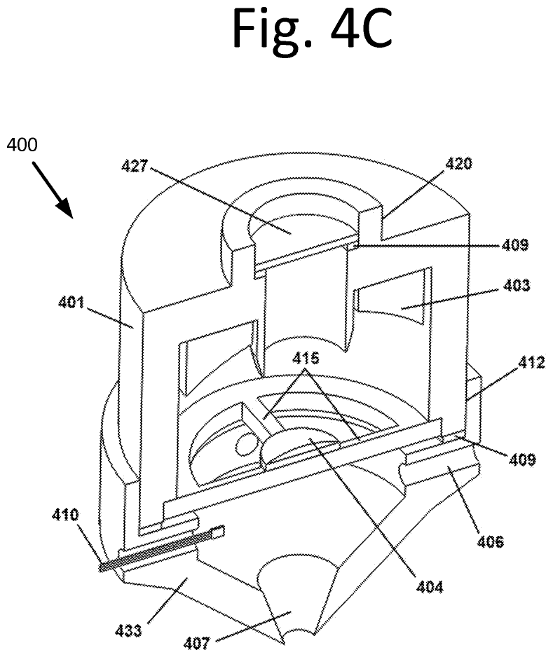

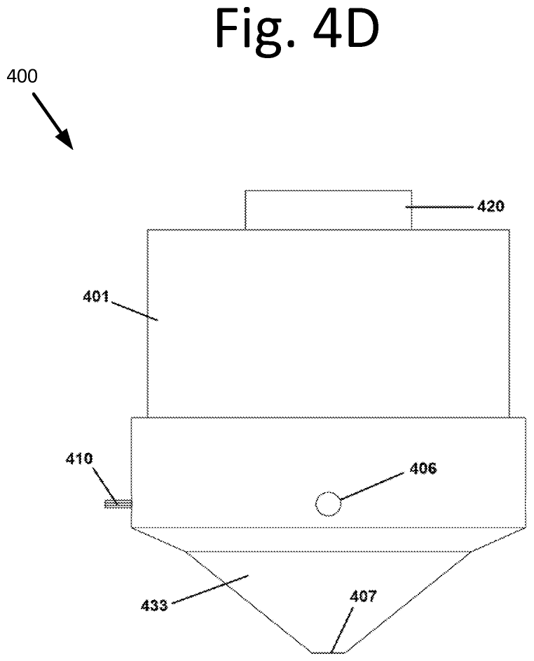

RFL NMAs, such as those depicted in FIGS. 2A-F, beneficially do not obstruct the RFL imaging and film thickness measurement beam 230 (not shown) with the nozzle 207 when conducting measurements because the nozzle is not in the path of the imaging and film thickness measurement beam(s). Depending on the design, RFL NMAs, such as those depicted in FIGS. 3 and 4, may obstruct or interfere with the RFL imaging and film thickness measurement beam(s) and lead to optical artifacts that deteriorate measurement quality and/or sensitivity if the artifacts are not corrected for. However, all RFL NMAs described in this application facilitate coaxial alignment between the film thickness measurement location and/or optical image with a normal impinging jet's stagnation point. Further, the RFL NMAs allow simultaneous monitoring and control of the impinging jet, and RFL and NMA components. These RFL NMA attributes are preferred for construction of RFL-TFV instruments as they enable routine, local physical property analysis of fluids resting on solid material surfaces, simplify the thin fluid film measurement process, expand measurement protocols, improve measurement accuracy and precision, and ease theoretical analysis.

When precise components and assembly methods are employed, all RFL NMAs described in this application facilitate coaxial alignment between the film thickness measurement location, optical image and impinging jet's stagnation point. The inclusion of XYZ positioners, for example, may ease component manufacturing precision requirements and facilitate alignment of these elements during RFL NMA design, construction and assembly. In an alternative embodiment, XY positioners on nozzle mount 208 moves the nozzle, and therefore, the jet's stagnation point relative to the film thickness measurement location and RFL NMA's optical image center.

Although not shown in the drawings, and depending on the embodiment, XYZ positioners are included on any RFL NMA sub-assembly, component or part. For purposes of explanation/clarification, there may be several alignment combinations, such as nozzle/nozzle, mount/spider, mount/nozzle, manifold/primary, and secondary mirrors, etc., all which have the same alignment purpose. During practice, a person of ordinary skill in the art may fix one, e.g., the primary mirror, and have XYZ translation for the spider mount, which has the secondary mirror and nozzle affixed. This is, however, a matter of application and design choice. With high precision components, such as machining and assembly, a person of ordinary skill in the art may construct the RFL NMA without positioners.

It should be noted that, in some embodiments, nozzle or nozzle mount may include a XYZ translation stage, which would allow precise positioning of the gas jet, and therefore, a stagnation point relative to the film thickness measurement location and optical image. This XYZ translation stage, in some embodiments, provides the preferred motion relative to the primary and secondary mirrors, whereas the XYZ translation positioners in the above paragraph moves the secondary mirror and nozzle relative to the primary mirror. It should be appreciated that XYZ positioners may be primarily used for alignment when manufacturing the RFL NMA. In such embodiments, adjustment to the positioner(s) is no longer required once alignment is complete (i.e., film thickness measurement location, optical image, nozzle center, stagnation point and nozzle standoff distance dₛ) are within manufacturing tolerances. If however, for example, the removable nozzle is switched with a different sized nozzle, then the XYZ positioners can be used again to bring the RFL NMA system into alignment. These embodiments may be applicable to one or more drawings described herein; however, this will be purely on design choice and application of RFL NMA.

FIGS. 3A-F are diagrams illustrating various views of RFL NMA 300, according to an embodiment of the present invention. Although RFL NMA 300 may be used with other reflective imaging lens designs, in these embodiments, RFL NMA 300 uses the general layout of a reverse Cassegrain-Schwarzschild reflective imaging objective as described in this literature. It is, therefore, understood some embodiments may be used with other RLF designs, including those that are not symmetric.

In some embodiments, such as that shown in FIGS. 3A-F, RFL NMA 300 uses a RFL upper assembly 325 comprised of upper body 301 with primary mirror 303 attached. Upper assembly 325 further includes RFL optical mount 320, NMA mounting features 312, cutout/hole/port features 311 and other general RFL features such as a stray light baffle. RFL NMA 300's nozzle manifold assembly 335 is comprised of lower body 302, which includes multi-purpose windows 327 and 328. Multi-purpose windows 327 and 328 are optically transparent over a desired wavelength range. In this embodiment, secondary mirror 304 is attached to the top-center of window 327, while nozzle 307 mounts to a hole at the bottom-center of window 328 with gasket 309 providing a seal. It should also be appreciated that secondary mirror 304 of NMA 335 may be directly, monolithically integrated or permanently installed/fixed to window 327. It should also be appreciated that, although NMA 335's nozzle 307 is depicted as removable from window 328, in certain embodiments, nozzle 307 may not be removable (i.e., nozzle 307 is directly, monolithically integrated or permanently installed/fixed to window 328). In this embodiment, nozzle 307 located within window 328, such as a single hole or multiple angled holes arranged in a radial pattern or arbitrary pattern, for example. In these embodiments, NMA 335 includes lower body 302 possessing various channels/holes/bores/cutouts, and features 306. When RFL NMA 300 is assembled, features 306 and 311 integrates sensors (e.g., temperature and pressure) and supply fluid (e.g., gas) into supply channel 316, which feeds gas (fluid) to nozzle 307 to form the impinging jet during use. To be clear, when windows 327 and 328 are mounted, with fasteners 329 and gaskets/seals 309, to NMA 335's lower body 302, supply channel 316 (or gap) is formed between two windows 327 and 328.

As shown in FIG. 3A, RFL NMA 300 includes an upper assembly 325 and lower nozzle manifold assembly 335. Here the lower body 302 of NMA 335 mounts onto or into upper body 301 of upper assembly 325 with features 312. Once these two assemblies 325 and 335 are mounted together, the RFL NMA 300 is complete and can be incorporated into scientific instruments like optical microscopes. It should be appreciated that the manner and features used to mount NMA 335 to upper assembly 325 is a matter of design choice. FIGS. 3B-F depict various views of the constructed RFL NMA 300. FIGS. 3B-C respectively depict cut-way and solid side-views illustrating the RFL NMA 300 features accessible after construction, including cutouts/ports/holes 306 and 311, which are used for sensor integration and to supply gas (fluid) for the impinging jet. Similarly, FIGS. 3D-F respectively depict solid-views with a top-perspective, bottom-perspective and bottom view illustrating the arrangement of the nozzle 307 attached to window 328 that is attached with fasteners 329 to lower body 302 of NMA 335.

Assembled NMA 335, in some embodiments, may mount onto or into upper body 301 to form the RFL NMA 300. Also in some embodiments, assembled NMA 335 possesses features and/or components including lower body 302, optically transparent windows 327 and 328, secondary mirror 304, nozzle 307, cutouts/ports/holes 306, supply channel 316, fasteners 329, gaskets/seals 309, gas (fluid) temperature sensor 310, features for integration (e.g. holes, bores, channels) and other sensors for monitoring-controlling NMA and jet properties (e.g. temperatures, nozzle pressure, barometric pressure, humidity etc.).

Depending on the embodiment, RFL NMA 300 is constructed as a single unit, two separate units, or three or more separate units each composed of specific features and components (i.e. sub-assemblies). For a single unit construction, NMA 335 features and components are incorporated directly into the RFL's body and structure. For clarity, RFL NMA 300 is depicted in FIG. 3A with two separate sub-assemblies (RFL upper assembly 325 and lower NMA 335). RFL NMA 300 may include the general components of a reflective imaging lens, including the housing, mirrors, reflective optics, stray light baffle, refractive optical elements for catadioptric (refractive-reflective) systems, upper body, primary mirror, windows, lower body, and secondary mirror to name a few.

NMA 335 of RFL NMA 300 includes features for mounting NMA 335 to upper assembly 325 as depicted in FIGS. 3B-F. NMA 335's lower body 302 includes a plurality of channels/holes/bores 306 for the jet supply inlet, integrating sensors and temperature-pressure monitoring and/or control. RFL NMA 300 also includes a nozzle 307, gaskets (including seals, glue, etc.) 309, and a jet temperature sensor 310. In this embodiment, gaskets/seals 309 ensure the gas/liquid supplied to RFL NMA 300 exits through the nozzle 307 to create the impinging jet. Jet temperature sensor 310 is shown in FIG. 3A as being placed through one of a plurality of channels/holes/bores 306 in the lower body 302. While not shown, it is understood that temperature sensor 310 is sealed into hole 306 after mounting to prevent the jet's gas supply from leaking through hole 306. One of ordinary skill in the art would recognize and consider the type of temperature sensor 310 and how it is integrated into NMA 335.

FIGS. 3A and 3B illustrate the use of a wired temperature sensor such as a thermocouple or platinum RTD. Depending on the sensor's mounting location and physical dimensions, the sensor and/or wires may interfere with the imaging and film thickness measurement beam(s), leading to optical artifacts that deteriorate image/measurement quality and/or sensitivity. For this reason, it is important to consider the physical location and dimensions of the temperature sensor when designing RFL NMA 300. Depending on the application, when a wired temperature sensor 310 cannot be used, a non-contact temperature sensor (e.g., pyrometer), which does not require placing sensor/wires into the optical path, is more appropriate. For example, a high emissivity material for temperature sensing purposes is placed inside gas channel 316 by attachment to window 327 on the opposite side of secondary mirror 304. A pyrometer may then measure the temperature of the high emissivity material, and therefore, the jet within channel 316. Similarly, the nozzle is made of or include a high emissivity material for temperature sensing purposes. It is important to recognize that precise measurement of the impinging jet's temperature with either a wired sensor or a pyrometer, one should consider thermal isolation of the sensor or high emissivity material when constructing the RFL NMA. As with other elements in these embodiments, RFL NMA's 300 and configuration is determined by the application's requirements (e.g., measurement accuracy, resolution and wavelength range).

RFL NMA 300 may include other sensors, such as those to measure and/or control the jet's nozzle pressure, humidity and/or flow rate. In an embodiment, RFL NMA 300 includes pressure sensors (not shown) to measure barometric and/or nozzle differential (gage) pressure. Pressure sensors may, for example, be placed in one of a plurality of port/holes 311 of upper body 301 and/or channels/holes/bores 306 in the lower body 302. In another embodiment, RFL NMA 300 includes infrared sensors, high emissivity material for non-contact temperature sensing, pyrometers and cameras etc., for measuring properties such as temperature of NMA components (jet, nozzle 307, lower body 302 etc.), atmosphere, and the sample being inspected/analyzed. Similar to RFL NMA 200 depicted in FIG. 2, the RFL NMA 300 includes a RFL mount for the optical system 320, RFL imaging and film thickness measurement beams (not shown), gas jet (not shown), and heating and cooling features (not shown). The heating and cooling of the RFL NMA 300 may be accomplished in various ways including, but not limited to, use of a removable heating and cooling jacket like that shown in FIG. 8, a surface mount heater/cooler like a Peltier device, integrating channels into the nozzle manifold body, or using external heating and cooling of the jet supply.

As depicted in FIGS. 3A-F, RFL NMA 300 use optically transparent windows 327 and 328 instead of a spider mount/vanes, similar to FIG. 2, to hold the secondary mirror 304 and nozzle 307. In this regard RFL NMA 300 beneficially do not obstruct the RFL imaging and film thickness measurement beam(s) with nozzle 307 or spider vanes as depicted in FIG. 2. While windows 327/328 offer the above benefit, the materials and thickness of the windows the RFL NMA 300 uses may affect the quality of the imaging and film thickness measurement beam(s).

When gas flows through the RFL NMA's nozzle, pressure/force is exerted on the windows which can lead to displacement/deformation/deflection of windows 327/328, and therefore, displacement of the secondary mirror 304 and nozzle 307. Due to this, one should consider the thickness and mechanical properties of windows 327/328 when designing the RFL NMA 300. One of ordinary skill in the art may also consider the window's optical properties, specifically the light transmission properties over an application's desired wavelength range. For example, the windows may strongly absorb light of certain wavelengths, attenuating light to an extent that precludes use of the RFL NMA 300 at certain wavelengths. Similarly, the refractive index of windows 327/328 may lead to optical artifacts that degrade image and film thickness measurement quality. In instances where these artifacts are non-negligible, the RFL primary and/or secondary mirrors, and/or additional optical elements can be designed and/or included to compensate for these types of artifacts. Alternatively, one or more of the transparent windows 327/328 can be replaced with a refractive lens for optical correction purposes. In this regard, the RFL NMA 300 would constitute a catadioptric (refractive-reflective) system which differs from RFL NMA 200 that only uses reflective mirrors (catoptric system). The exact design and integration of the various RFL NMA 300 elements, including primary and secondary mirrors, windows, lenses, nozzle, sensors, and wire dimensions, etc., require consideration of, but not limited to, the desired optical performance, volumetric flow rates, thermal isolation, and measurement (e.g., viscosity, film thickness) dynamic range. One of ordinary skill in the art may consider these various aspects when designing and, selecting sensors and materials to construct RFL NMA 300 for a particular application.

FIGS. 4A-D are diagrams illustrating various views of RFL NMA 400, according to an embodiment of the present invention. Although RFL NMA 400 may be used with other reflective imaging lens designs, RFL NMA 400 in these embodiments uses the general layout of a reverse Cassegrain-Schwarzschild reflective imaging objective. It should be appreciated that this configuration can be used with other RLF designs, including those that are not symmetric.

In some embodiments, as depicted in FIGS. 4A-D, RFL NMA 400 uses RFL upper assembly 425 comprised of upper body 401, primary mirror 403 attached to upper body 401, secondary mirror 404 attached to spider mount/vanes 415 mounted to upper body 401, and other general RFL features such as a stray light baffle (not labeled). Upper assembly 425 further includes RFL optical mount 420, optically transparent window 427, gaskets/seals/glue 409, features to mount NMA 435, and cutout/hole/port features (not shown) to integrate sensors for monitoring/controlling properties of RFL NMA 400. While not shown, RFL upper assembly 425 may also include additional gaskets, seals and glues to ensure gas supplied to assembled RFL NMA 400 only exits nozzle 407 in nozzle manifold assembly 435.

RFL NMA 400 may include a lower nozzle manifold assembly 435, which is comprised of nozzle manifold 433. Nozzle manifold 433 may include nozzle 407, gaskets/seals/glue 409, various channels/holes/bores/cutouts 406, mounting features 412, and gas (fluid) temperature sensor 410. In these embodiment, and unlike RFL NMA 200 of FIGS. 2A-F, spider mount/vanes 415 do not possess features (e.g. channels, holes, bores, etc.) to flow the gas (fluid) from an external gas supply to the nozzle. For RFL NMA 400, nozzle manifold 433 of lower nozzle manifold assembly 435 possesses features 406 to supply gas to the nozzle 407 to create the impinging jet. It should also be appreciated that, although nozzle 407 is depicted as monolithically integrated into nozzle manifold 433, in certain embodiments, nozzle 407 may be a separate, removable part that mounts to nozzle manifold 433. Furthermore, whether monolithically integrated or as a separate part, nozzle 407 may be made with a single orifice, as shown, or with multiple holes arranged in a radial or arbitrary pattern, for example. In these embodiments, NMA 435 includes nozzle manifold 433 possessing nozzle 407 and various features (e.g., channels/holes/bores/cutouts) 406. When RFL NMA 400 is assembled, features 406 integrate sensors (e.g. temperature and pressure) and supply fluid (e.g., gas) to RFL NMA 400 that feed to nozzle 407 to form the impinging jet during use. To be clear, when RFL NMA 400 is assembled with the appropriate fasteners and gaskets/seals/glues, the supplied gas (fluid) exits nozzle 407 of RFL NMA 400.

As shown in FIG. 4A, RFL NMA 400 includes an upper assembly 425 and lower nozzle manifold assembly 435. Here, lower body 433 of NMA 435 mounts onto or into upper body 401 of upper assembly 425 with features 412. Once these two assemblies, upper assembly 425 and lower nozzle manifold assembly 435, are mounted together, RFL NMA 400 is complete and is incorporated into scientific instruments such as optical microscopes. It should be appreciated that the manner and features used to mount NMA 435 to upper assembly 425 is a matter of design choice. FIGS. 4B-D depict various views of the constructed RFL NMA 400 according to embodiments of the invention. FIGS. 4B-D respectively depict side cut-way, side-perspective and solid side views illustrating the various features of RFL NMA 400, including cutouts/ports/holes 406 that are used for sensor integration and to supply gas (fluid) for the impinging jet. Furthermore, FIGS. 4B-C illustrate the relative arrangement of nozzle 407 to the primary 403 and secondary mirrors 404 within upper assembly 425.

Amongst other things, assembled NMA 435 mounts onto or into upper body 401 of upper assembly 425 to form the RFL NMA 400. NMA 435 possesses features and/or components including nozzle manifold 433 that incorporates nozzle 407, gaskets/seals/glue 409, various channels/holes/bores/cutouts 406, mounting features 412, gas (fluid) temperature sensor 410, features for integration (e.g., holes, bores, channels) and other sensors for monitoring-controlling NMA and jet properties (e.g., temperatures, nozzle pressure, barometric pressure, humidity, etc.). In some embodiments, NMA 435 mounting to upper assembly 425 is a matter of design choice and application needs. Although NMA 435 is depicted as being removable from upper assembly 425, NMA 435, in certain embodiments, may not be removable (i.e., NMA 435 is permanently installed/fixed to upper assembly 425). In some embodiments, a removable NMA 435 is beneficial, because NMA 435 allows transfer of NMA 435 from one RFL upper assembly 425 to another.

Depending on the embodiment, RFL NMA 400 may be constructed as a single unit, two separate units, or three or more separate units each composed of specific features and components (i.e. sub-assemblies). For a single unit construction, NMA 435 features and components are incorporated directly into the RFL's body and structure. For clarity, RFL NMA 400 is depicted in FIG. 4A with two separate sub-assemblies (i.e., RFL upper assembly 425 and lower NMA 435). RFL NMA 400 may include the general components of a reflective imaging lens, including the housing, mirrors, reflective optics, stray light baffle, RFL optical mount 420, refractive optical elements for catadioptric (refractive-reflective) systems, upper body 401, primary mirror 403, spider mount/vanes 415, and secondary mirror 304 to name a few.

NMA 435 of RFL NMA 400 includes features for mounting NMA 435 to upper assembly 425 as depicted in FIGS. 4B-D. NMA 435's nozzle manifold 433 includes a plurality of channels/holes/bores 406 for the jet supply inlet, integrating sensors and temperature-pressure monitoring and/or control. RFL NMA 400 also includes a nozzle 407, gaskets (including seals, glue, etc.) 409, and a jet temperature sensor 410. In this embodiment, gaskets/seals/glue 409, as well as others not shown, to ensure the gas/liquid supplied to RFL NMA 400 exits through nozzle 407 to create the impinging jet used for sample inspection-analysis.

Jet temperature sensor 410 of FIG. 4A-D is placed through one of a plurality of channels/holes/bores 406 in nozzle manifold 433. While not shown, it is understood that temperature sensor 410 is sealed into hole 406 after mounting to prevent the jet's gas supply from leaking through hole 406. It is important to recognize and consider the type of temperature sensor 410 and how it is integrated into the NMA 435. FIGS. 4A-D illustrate the use of a wired temperature sensor such as a thermocouple or platinum RTD. Depending on the sensor's mounting location and physical dimensions, the sensor and/or wires can interfere with the imaging and film thickness measurement beam(s), leading to optical artifacts that deteriorate image/measurement quality and/or sensitivity.

It is, therefore, important to consider the physical location and dimensions of the temperature sensor when designing the RFL NMA 400. For some embodiments, a wired temperature sensor 410 cannot be used and a non-contact temperature sensor (e.g. pyrometer) that does not require placing sensor/wires into the optical path may be more appropriate. For example, a high emissivity material for temperature sensing purposes may be attached to spider mount/vanes 415 on the opposite side of secondary mirror 404. A pyrometer may measure the temperature of the high emissivity material, and therefore, the gas jet. Similarly, the nozzle may be made of or include a high emissivity material for temperature sensing purposes. It is important to recognize that precise measurement of the impinging jet's temperature with either a wired sensor or a pyrometer, one should consider thermal isolation of the sensor or high emissivity material when constructing the RFL NMA. As with other elements, the RFL NMA's design and configuration is determined by the application's needs and requirements (e.g., measurement accuracy, resolution and wavelength range).

RFL NMA 400 may include other sensors, such as those to measure and/or control the jet's nozzle pressure, humidity and/or flow rate. In one embodiment, RFL NMA 400 includes pressure sensors (not shown) to measure barometric and/or nozzle differential (gage) pressure. Pressure sensors, for example, may be placed in one of a plurality of port/holes (not shown) of upper body 401 and/or channels/holes/bores 406 in nozzle manifold 433. In another embodiment, RFL NMA 400 includes infrared sensors, high emissivity material for non-contact temperature sensing, pyrometers and cameras etc. for measuring properties like the temperature of NMA components (e.g., jet, nozzle 307, nozzle manifold 433 etc.), atmosphere, and the sample being inspected/analyzed. Similar to RFL NMAs 200 and 300 depicted in FIGS. 2-3, RFL NMA 400 includes RFL optical system mount 420, RFL imaging and film thickness measurement beams (not shown), gas jet (not shown), and heating and cooling features (not shown). The heating and cooling of RFL NMA 400 may be accomplished in various ways including use of a removable heating and cooling jacket such as that shown in FIG. 8, a surface mount heater/cooler (e.g., a Peltier device), integrating channels into the nozzle manifold body, or using external heating and cooling of the jet supply.

As depicted in FIGS. 4A-D, RFL NMA 400 use optically transparent window 427, nozzle manifold 433 and various gaskets, seals and glues to ensure gas supplied to the assembled RFL NMA 400 only exits nozzle 407 in nozzle manifold assembly 435. In this regard, RFL NMA 400 beneficially does not obstruct the RFL imaging and film thickness measurement beam(s) with nozzle manifold 433 or nozzle 407 over a certain wavelength range. To be clear, the imaging and film thickness measurement beam(s) pass through nozzle 407 unobstructed. While window 427 offers the above benefit, the material and thickness of window 427, can affect the quality of the imaging and film thickness measurement beam(s). When gas flows through RFL NMA's nozzle, pressure/force is exerted on the window that can lead to displacement, deformation and/or curvature of window 427, potentially introducing undesirable optical artifacts of the imaging and film thickness measurement beam(s). Due to this, a person of ordinary skill in the art would consider the thickness and mechanical properties of window 427 when designing and constructing RFL NMA 400.

A person of ordinary skill in the art would also consider the window's optical properties, specifically the light transmission properties over an application's desired wavelength range. For example, window 427 may strongly absorb light of certain wavelengths, and therefore, attenuate light to an extent that precludes use of RFL NMA 400 at certain wavelengths. Similarly, the refractive index of windows 427 leads to optical artifacts that degrade image and measurement (e.g. film thickness) quality. In instances where these artifacts are non-negligible, the RFL primary and/or secondary mirrors, and/or additional optical elements are designed and/or included to compensate for these types of artifacts. Alternatively, window 427 may be replaced with a refractive lens for optical correction purposes. In this regard, RFL NMA 400 may constitute a catadioptric (refractive-reflective) system, which differs from RFL NMA 200, uses reflective mirrors (catoptric system).

In some embodiments, it should be appreciated that integration of window 427 into RFL NMA 400 is a matter of design choice and application needs. Although window 427 is depicted as being removable from upper assembly 425's optical mount 420, in certain embodiments window 427 may not be removable (i.e., window 427 is permanently installed/fixed to optical mount 420). In some applications, a removable window 427 is beneficial. For example, the combination of having removable window 427 and removable NMA 435, as described earlier, allows transfer of window 427 and/or NMA 435 from one RFL upper assembly 425 to another. Additionally, another benefit of a removable window 427 is the elimination of optical artifacts the window introduces to the imaging and measurement beams.

The exact design, construction and integration of the various RFL NMA 400 elements, including primary and secondary mirrors, window(s), lenses, nozzle, seals, fasteners, sensors, and wires dimensions, etc., require consideration of, but not limited to, the desired optical performance, volumetric flow rates, thermal isolation, and measurement dynamic range (e.g., viscosity, film thickness). One with ordinary skill in the art should consider these various aspects when designing and selecting sensors and materials to construct RFL NMA 400 for a particular application.

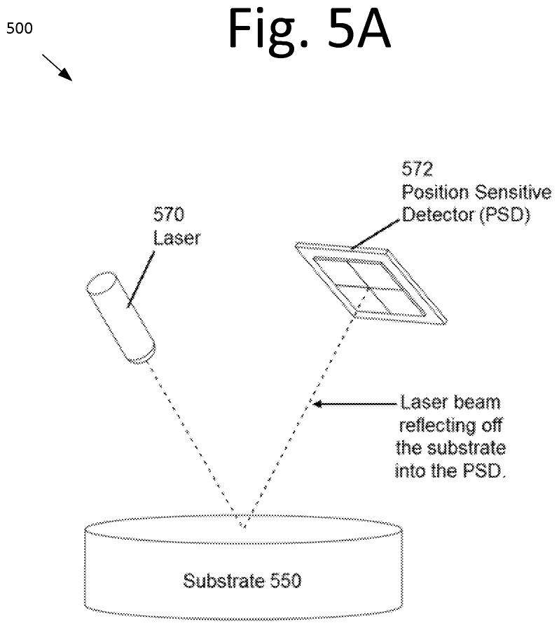

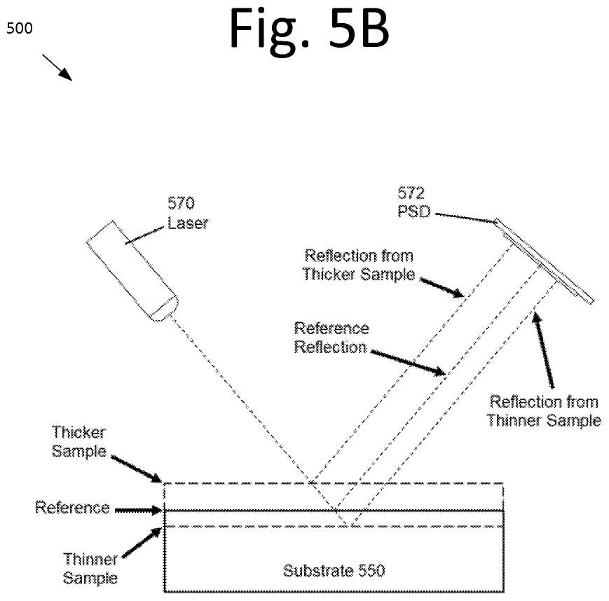

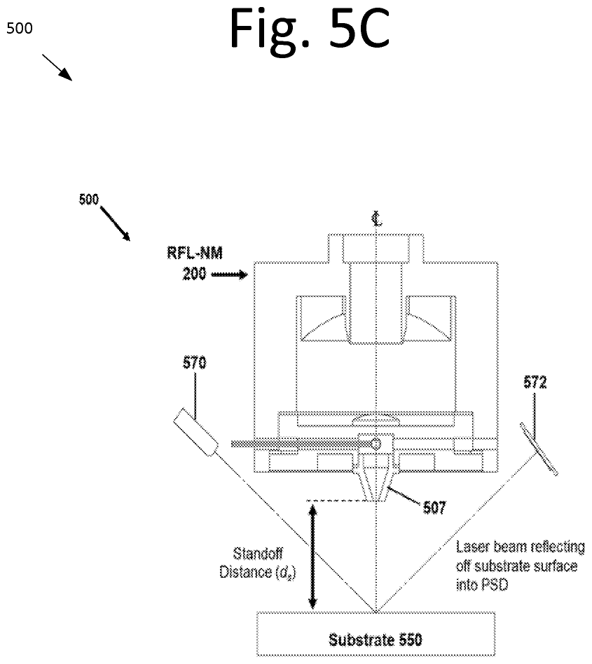

FIGS. 5A-C are diagrams illustrating a configuration 500 for triangulating with a position sensitive detector (PSD) 572, according to an embodiment of the present invention. In this embodiment, laser 570 transmits a laser beam towards substrate 550, in which the laser beam reflects off substrate 550's surface into PSD 572. Substrate 550 and sample (not shown) may be one in the same depending on the embodiment. It should be noted that, depending on the thickness of the sample, the position of reflection into PSD 572 may change. See, for example, FIG. 5B. More specifically, PSD 572 measures a high laser position for a substrate thicker than the reference substrate. This reading instructs the user to move RFL NMA or substrate vertically until the PSD reading matches the value recorded during background measurement with the reference sample. For instance, in FIG. 5B, PSD 572 may record “0” during background measurements with the reference sample. Now, when the thicker sample is measured, PSD 572 records a high value. The user may move RFL NMA or sample vertically until PSD 572 sees “0” to match the reference value. Once matched to “0”, the user proceeds to make optical, film thickness and viscosity measurements.

As shown in FIG. 5C, in this embodiment, laser 570—PSD 572 triangulation is used to monitor and control RFL NMA 200's nozzle 507 position relative to substrate 550's surface. This triangulation ensures the standoff distance (dₛ) is highly repeatable when making film thickness, TFV viscosity and other measurements. FIG. 5C illustrates a general arrangement and alignment of the laser 570—PSD 572 about RFL NMA 300 and the centerline that corresponds to the RFL NMA's optical axis, image center, focal point, measurement location and impinging jet stagnation point. Laser 570 and PSD 572, in some embodiments, are incorporated into RFL NMA 200 in various ways including permanent integration or as a removable component, which can be used with different RFL NMAs such as those with different magnifications. Whether permanently integrated or as a removable component, the fixture for laser 570 and PSD 572 includes the ability to adjust their position, e.g., with angle and xyz positioners, so that the point where the laser spot reflects off the substrate coincides with the centerline, which corresponds to the RFL NMA's optical axis, image center and focal point, measurement location and jet stagnation point. Laser 570 and PSD 572 for triangulation may also be integrated into the TFV instrument, e.g., on structural components that RFL NMA is mounted on in the TFV.

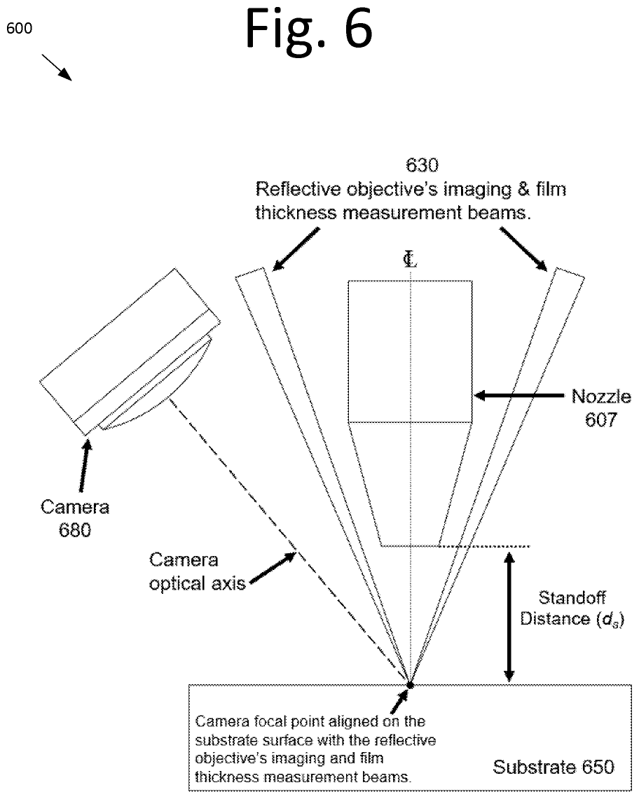

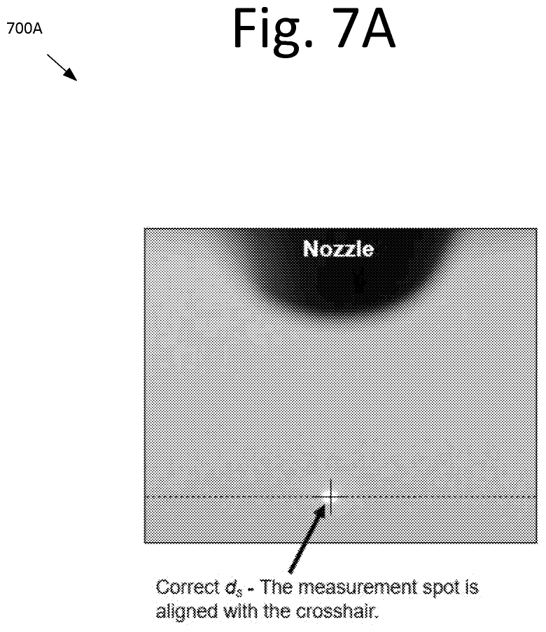

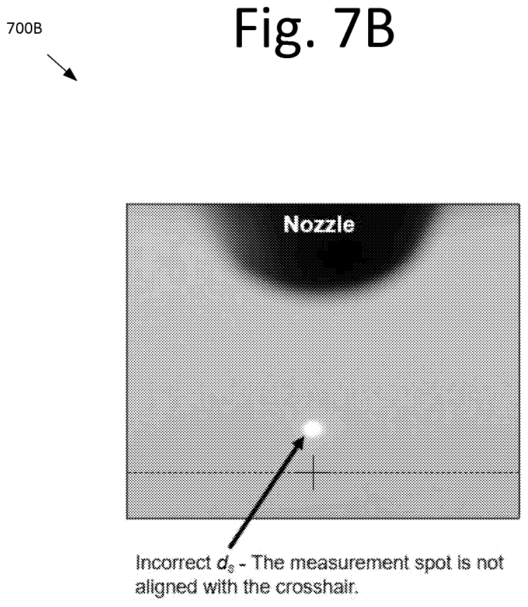

FIG. 6 is a diagram illustrating a camera triangulation schematic 600, according to an embodiment of the present invention. In this embodiment, a camera 680 is arranged at an oblique angle relative to RFL NMA 200. When at the correct standoff distance dₛ, camera 680's focal point and RFL NMA 200's measurement spot are aligned on the surface of substrate 650. See, for example, FIGS. 7A and 7B, which show images 700A and 700B illustrating use of camera triangulation, according to an embodiment of the present invention. In image 700A, the correct standoff distance is shown, i.e., at the correct standoff distance dₛ, the camera focal point and the measurement spot are aligned on the substrate surface. In image 700B, the incorrect standoff distance dₛ is shown, i.e., the measurement spot is not aligned with the crosshair. In this case, when standoff distance dₛ is incorrect, RFL NMA or sample is moved vertically to align the measurement spot with the crosshair. Camera 680 may be incorporated into RFL NMA 200 in various ways including permanent integration or as a removable component that can be used with different RFL NMAs like those with different magnifications. Whether permanently integrated or as a removable component, camera 680 may include the ability to adjust its position, say with angle and xyz positioners, so camera 680's focal point and image center coincides with the centerline, which corresponds to the RFL NMA's optical axis, image center and focal point, measurement location and jet stagnation point. Camera 680 may also be integrated into the TFV instrument, e.g., on structural components that the RFL NMA is mounted to.

FIGS. 8A-D are diagrams illustrating various views of a RFL NMA 800 with the integrated heat-cooling features for temperature control, according to an embodiment of the present invention. In some embodiments, the temperature of RFL NMA, and therefore, the gas jet is controlled in various ways including direct integration of heaters/coolers into RFL NMA, placement of RFL NMA based instruments in a temperature-controlled environment, external conditioning of the gas jet supply, and/or use of removable heating/cooling elements (e.g., device 815).

In certain embodiments, FIG. 8A shows a removable heater/cooler (RHC) 815 with integrated features for heating-cooling that mounts with clamping fastener 804 to a RFL NMA. As shown in FIG. 8B-D, RFL NMA 800 possesses similar features as RFL NMA 200 that are included by reference, but also includes RHC 815 possessing inlet outlet ports and channels 805 to supply temperature-controlled media, e.g. fluid, from a recirculating heater-chiller, for example. A removable RHC 815 may be beneficial in applications where different RFL NMAs are used, allowing transfer of the RHC 815 from one RFL NMA to another. It should be appreciated that the RFL NMA 800 type shown in these embodiments, with heating and cooling, may also be combined with the laser-PSD and camera triangulation, as shown in FIGS. 5-7. Similarly, RHC 815 may be used with other RFL NM designs, including RFL NM 300 and 400 to name a couple.

In these embodiments, RHC 815 includes a plurality of inlet ports and outlet ports, as well as channels 805, for heating and cooling media. Similar to RFL NMA 200, there are also a plurality of channels, holes and bores 806 used for the jet supply inlet, integrating sensors and temperature monitoring-control. RFL NMA 800 further includes nozzle 207, gaskets (seals, glues, etc.) 809 and jet temperature sensor 210. It should be appreciated that, although nozzle 207 for NMAs are removable, in certain embodiments, nozzle 207 may be permanent (i.e., integrated into the NMA as a single unit).

FIGS. 8A-B respectively show solid and cutaway views with the RFL NMA 200 and RHC 815 as separate components prior to assembling into or onto the RFL NMA 800. FIGS. 8C-D respectively show cutaway and solid views after assembling RFL NMA 800. As with RFL NMAs 200, 300 and 400, RFL NMA 800 may include other sensors such as those to measure and/or control the jet's nozzle pressure, humidity and/or flow rate. In an embodiment, RFL NMA 800 includes pressure sensors (not shown) to measure barometric and/or nozzle differential pressure. Pressure sensors can, for example, be placed in one of a plurality of channels/holes/bores 806 in the RHC 815 and connect to RFL NMA 200. In another embodiment, RFL NMA 800 can include infrared sensors, pyrometers, and cameras, etc., (not shown) for measuring properties like the temperature of the jet and/or nozzle, atmosphere, and sample being inspected and analyzed.

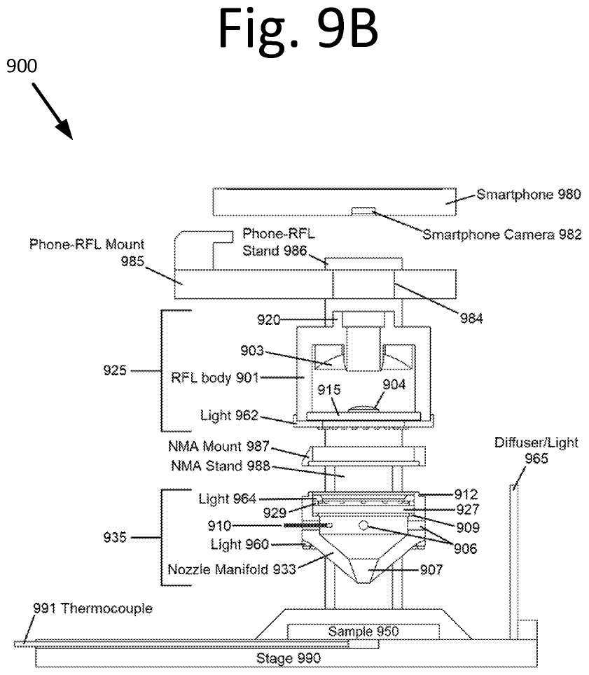

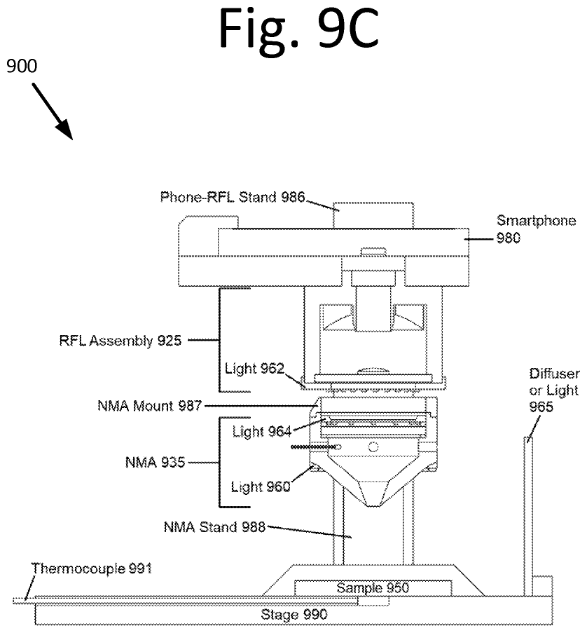

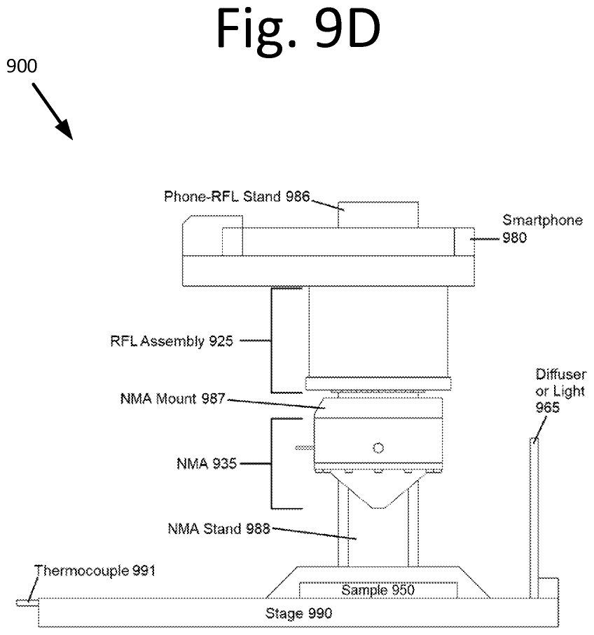

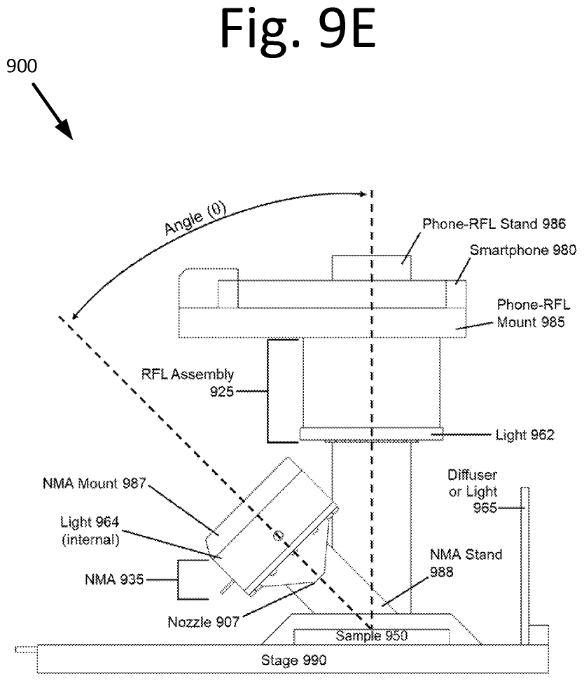

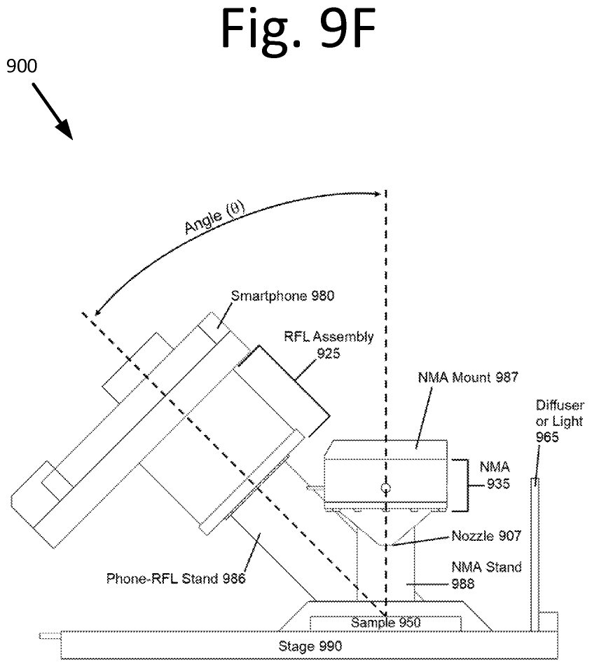

FIGS. 9A-F are diagrams illustrating various views of a modular RFL NMA 900 coupled to a smartphone 980 or other sensing devices, according to an embodiment of the present invention. In some embodiments, RFL NMA 900 is coupled to microscopes, machine vision zoom lens systems and retail imaging products such smartphones 980, and USB devices, such as cameras, endoscopes and microscopes. In some instances, the RFL used in the RFL NMA 900 is a lens for magnification. In other instances, RFL NMA 900 may exclude the RFL and therefore the NMA 935 couples directly to the smartphone device 980. In other embodiments, RFL NMA 900 is coupled to a smartphone 980 for inspection and analysis of samples 950 (including sample surfaces coated with fluid films).

In some embodiments, modular RFL NMA 900 is made with a smartphone 980 using a phone RFL mount 985, phone RFL stand 986, NMA mount 987, NMA stand 988 and stage 990. In these embodiments, two separate mounts and stands are used, i.e., phone RFL mount/stand 985/986 and NMA mount/stand 987/988. Phone RFL mount 985 is used to couple the smartphone and RFL assembly 925, where in some embodiments RFL assembly 925 is a lens for inspection and/or imaging of the sample 950. NMA mount 987 and stand 988 are used to hold and position the NMA 935 above sample 950 placed on stage 990. The stage 990, phone RFL mount 985, phone RFL stand 986, NMA mount 987 and NMA stand 988 may include a plurality of features and sensors, such as fixtures to hold and position components and sample, temperature sensors like thermocouple 991, infrared sensors, pyrometers and/or cameras, etc. (not shown) for measuring properties like the temperature of the jet, atmosphere and sample 950 being inspected and analyzed.

Depending on the embodiment, RFL NMA 900 may be constructed as a single unit, two separate units, or three or more separate units each composed of specific features and components (i.e. sub-assemblies) pending application. For example, NMA 935 features and components can be incorporated directly into RFL assembly 925's body and structure which reduces the number of RFL NMA 900 sub-assemblies. For clarity the RFL NMA 900 is depicted in FIGS. 9A-F with six sub-assemblies (smartphone 980, phone-RFL mount/stand 985/986, NMA mount/stand 987/988, RFL assembly 925, NMA 935 and stage 990). The RFL NMA 900 may include the general components of a reflective imaging lens, including the housing, mirrors, reflective optics, stray light baffle, RFL optical mount, refractive optical elements for catadioptric (refractive-reflective) systems, upper body, primary mirror, spider mount/vanes, and secondary mirror to name a few.

As depicted in FIGS. 9A-F, the invention's modular RFL NMA 900 configuration allows smartphones, RFL and NMA axes to be positioned relative to each other and the sample/stage 950/990. In this embodiment, the smartphone 980 and RFL assembly 925 are placed on phone RFL mount 985 which is attached to the phone RFL stand 986 that allows both vertical and angular (θ) positioning relative to the NMA 935 and sample/stage 950/990. FIGS. 9D/F illustrate normal and oblique angular positioning of the RFL assembly 925 with the RFL NMA 900. Similarly, the NMA 935 is placed on NMA mount 987 which is attached to NMA stand 988 that also allows vertical and angular (θ) positioning of NMA 935, and therefore the nozzle axis, relative to the RFL assembly 925 and the sample/stage 950/990. FIGS. 9D/E illustrate normal and oblique angular positioning of the NMA 935 with the RFL NMA 900. As depicted in FIGS. 9E and F, when conducting imaging-analysis and/or using an oblique impinging jet, a light source or light diffuser 965 attached to stage 990 or elsewhere may beneficially improve inspection clarity and/or resolution. It is understood that there are various design configurations for the various components of RFL NMA 900, where for example mounts 985/987 and stands 986/988 and stage can be designed to allow variable, multi-axis positioning (θ, X, Y, Z) of any component relative to each other.

As depicted in FIGS. 9A-F the two separate mounts (e.g., phone RFL mount 985 and NMA mount 987) are able to move vertically on their respective stands. This allows both the smartphone 980-RFL assembly 925 and NMA 935 to be moved independently so that the surface of sample 950 can be brought into focus and measurements made at the correct/desired nozzle 907 standoff distance (dₛ) as discussed previously. The phone RFL mount 985, phone RFL stand 986, NMA mount 987, NMA stand 988, and stage 990 may include physical markings, like a ruler, or position encoders to facilitate accurate vertical positioning and sample placement. FIG. 9A shows a 3D perspective of the constructed RFL NMA 900, while FIG. 9B shows a 2D sectioned front view with the various components/assemblies separated for clarity. FIGS. 9C-F show 2D views of the constructed RFL NMA 900 with variable angular arrangements of the RFL assembly 925 and NMA 935.

FIG. 9B shows RFL NMA 900 with components separated prior to assembly, and therefore prior to coupling smartphone 980 (or other portable device) possessing camera (or sensor) 982 to RFL assembly 925 and NMA 935 according to an embodiment of the present invention. In some embodiments, the smartphone 980 is attached to phone RFL mount 985 that possesses hole mounting feature 984. Here, feature 984 provides a clear path for the optical (or other) signal during the measurement process and also acts as a feature to mount the RFL assembly 925. In this manner, during assembly the smartphone camera 982 can be positioned-aligned with center axis of the RFL's imaging-measurement beams (not shown), and the NMA 935's nozzle and impinging jet (not shown).

As depicted in FIG. 9B, RFL assembly 925 uses the general layout of a reverse Cassegrain-Schwarzschild reflective imaging objective as described in literature. It is therefore understood the invention can be used with other RFL designs, including those that are not symmetric. RFL assembly 925 is comprised of upper body 901, primary mirror 903 attached to upper body 901, secondary mirror 904 attached to the spider mount/vanes 915 mounted to upper body 901, and other general RFL features like a stray light baffle (not labeled). RFL assembly 925 further includes RFL optical mount 920, and removable light/radiation source 962 attached to RFL body 901. Here, light/radiation source 962 is shown attached to the exterior of RFL body 901, which can be used in conjunction with the RFL's imaging and film thickness measurement beams that are typically supplied using epi-illumination techniques. For example, light/radiation source 962 may emit a broadband of wavelengths, tunable wavelength(s) or specific wavelength of light/radiation such as an excitation wavelength used in fluorescent spectroscopy or for light/radiation exposure testing. Depending on the embodiment, installing multiple light/radiation sources may be beneficial, including for example, a light/radiation source placed within stage 990 for inspection of transparent samples (not shown).

While not shown and pending application, the RFL assembly 925 may also include gaskets/seals/glue, additional optics for coupling to the smartphone device's camera 982, epi-illumination port or module, features for direct mounting of NMA 935, and cutout/hole/port features to integrate sensors for monitoring/controlling properties of the RFL NMA 900. In this embodiment, and unlike RFL NMA 200 depicted in FIGS. 2A-F, the spider mount/vanes 915 do not possess features (e.g. channels, holes, bores etc.) to flow the gas (fluid) from an external gas supply to the nozzle 907. For the RFL NMA 900, the nozzle manifold 933 of nozzle manifold assembly 935, possesses features 906 to supply gas to the nozzle 907 to create the impinging jet. To be clear, when the NMA 935 is assembled with the multi-function window 927 installed to manifold 933, features 906 are used to integrate sensors (e.g. temperature and pressure) and supply fluid (e.g. gas) that feeds into and exits NMA 935 through nozzle 907 to form the impinging jet. It should also be appreciated that although NMA 935's nozzle 907 is depicted as monolithically integrated into nozzle manifold 933, in certain embodiments, nozzle 907 may be a separate, removable part that mounts to the nozzle manifold 933. In this respect, a removable nozzle 907 can offer the benefit of being able to switch nozzles with different features (e.g. nozzle orifice dimensions and/or geometries). Furthermore, whether monolithically integrated or as a separate part, nozzle 907 can be made with a single orifice as shown or multiple holes arranged in a radial or arbitrary pattern for example.

In some embodiments, the assembled NMA 935 mounts onto or into NMA mount 987 using features like mount 912. Amongst other things the assembled NMA 935 possesses features and/or components including nozzle manifold 933 that incorporates nozzle 907, gaskets/seals/glue 909, various channels/holes/bores 906, mounting features 912, gas (fluid) temperature sensor 910, optically transparent window 927, window fastener 929, lights/radiation sources 960 and 964, features for integration (e.g. holes, bores, channels) and other sensors (not shown) for monitoring-controlling NMA and jet properties (e.g. temperatures, nozzle pressure, barometric pressure, humidity etc.). In some embodiments, it should be appreciated that the manner NMA 935 mounts to NMA mount 987 is a matter of design choice and application needs. Although NMA 935 is depicted as being removable from NMA mount 987, in certain embodiments NMA 935 may not be removable (i.e., it is permanently installed/fixed to NMA mount 987). In some embodiments, a removable NMA 935 is beneficial, as it allows installing a different NMA type to NMA mount 987, such as one with a different nozzle geometry/dimension.

NMA 935's nozzle manifold 933 includes a plurality of channels/holes/bores 906 for the jet supply inlet, integrating sensors and temperature-pressure monitoring and/or control. NMA 935 also includes a nozzle 907, gaskets (including seals, glue, etc.) 909, and a jet temperature sensor 910. In this embodiment, gaskets/seals/glue 909 as well as others not shown, ensure the gas/liquid supplied to the NMA 935 exits through the nozzle 907 to create the impinging jet used for sample inspection-analysis. Jet temperature sensor 910 is shown in FIGS. 9A-F as being placed through one of a plurality of channels/holes/bores 906 in the nozzle manifold 933. While not shown, it is understood that temperature sensor 910 is sealed into hole 906 after mounting to prevent the jet's gas supply from leaking through hole 906. It is important to recognize and consider the type of temperature sensor 910 and how it is integrated into the NMA 935. FIGS. 9A-F illustrate the use of a wired temperature sensor like a thermocouple or platinum RTD. Depending on the sensor's mounting location and physical dimensions, the sensor and/or wires can interfere with the imaging and film thickness measurement beam(s), leading to optical artifacts that deteriorate image/measurement quality and/or sensitivity. It is therefore important to consider the physical location and dimensions of the temperature sensor when designing the RFL NMA 900. For some applications, a wired temperature sensor like 910 cannot be used and a non-contact temperature sensor (e.g. pyrometer) that does not require placing sensor/wires into the optical path is more appropriate. For example, a high emissivity material for temperature sensing purposes can be attached to NMA 935. A pyrometer can then be used to measure the temperature of the high emissivity material and therefore the gas jet. Similarly, the nozzle can be made of or include a high emissivity material for temperature sensing purposes. It is important to recognize that precise measurement of the impinging jet's temperature with either a wired sensor or a pyrometer one should consider thermal isolation of the sensor or high emissivity material when constructing the RFL NMA 900. As with other elements of the invention, the RFL NMA's design and configuration is determined by the application's needs and requirements (e.g., measurement accuracy, resolution and wavelength range).

RFL NMA 900 can include other sensors, such as those to measure and/or control the jet's nozzle pressure, humidity and/or flow rate. In one embodiment, RFL NMA 900 includes pressure sensors (not shown) to measure barometric and/or nozzle differential (gage) pressure. Pressure sensors can, for example, be placed in one of a plurality of channels/holes/bores 906 in the nozzle manifold 933. In another embodiment, RFL NMA 900 can include infrared sensors, high emissivity material for non-contact temperature sensing, pyrometers and cameras etc. for measuring properties like the temperature of NMA components (jet, nozzle 907, nozzle manifold 933 etc.), atmosphere, and the sample being inspected/analyzed. Like RFL NMAs 200, 300 and 400 depicted in FIGS. 2-4, the RFL NMA 900 further includes the RFL optical system mount 920, RFL imaging and film thickness measurement beams (not shown), gas jet (not shown), and heating and cooling features (not shown). The heating and cooling of the RFL NMA 900 may be accomplished in various ways including use of a removable heating and cooling jacket like that shown in FIG. 8, a surface mount heater/cooler like a Peltier device, integrating channels into the nozzle manifold body, or using external heating and cooling of the jet supply.

As depicted in FIGS. 9B-C, NMA 935 uses a window 927 that is optically transparent over a desired wavelength range, nozzle manifold 933 and various gaskets, seals and glues to ensure gas supplied to the assembled NMA 935 exits nozzle 907. In this regard RFL NMA's using NMA 935 beneficially do not obstruct the RFL imaging and film thickness measurement beam(s) with nozzle manifold 933 or nozzle 907 over a certain wavelength range. To be clear, the imaging and film thickness measurement beam(s) pass through the nozzle 907 and window 927 unobstructed. While window 927 offers the above benefit, the material and thickness of window 927 the NMA 935 uses can affect the quality of the imaging and film thickness measurement beam(s). When gas/liquid flows through the NMA 935's nozzle, pressure/force is exerted on the window that can lead to displacement, deformation and/or curvature of the window 927, potentially introducing undesirable optical artifacts of the imaging and film thickness measurement beam(s). Due to this, one should consider the thickness and mechanical properties of window 927 when designing and constructing NMAs with windows like shown in NMA 935. One should also consider the window's optical properties, specifically the light/radiation transmission properties over an application's desired wavelength range. For example, window 927 may strongly absorb light of certain wavelengths and therefore attenuate light to an extent that precludes use of the RFL NMA 900 at certain wavelengths. Similarly, the refractive index of windows 927 can lead to optical artifacts that degrade image and measurement (e.g. film thickness) quality. In instances where these artifacts are non-negligible, the RFL primary and/or secondary mirrors, and/or additional optical elements can be designed and/or included to compensate for these types of artifacts. Alternatively, window 927 can be replaced with a refractive lens for optical correction purposes. In this regard the RFL NMA 900 would constitute a catadioptric (refractive-reflective) system which differs from the RFL NMA 200 that only uses reflective mirrors (catoptric system).

FIGS. 9B-C show the location of light/radiation sources 960 and 964 respectively attached to the exterior and interior of the nozzle manifold 933, both of which can be used in conjunction with the RFL's imaging and film thickness measurement beams. For example, light/radiation sources 960 and/or 964 may be designed to emit a broadband of wavelengths, tunable wavelength(s) or specific wavelength of light such as an excitation wavelength used in fluorescent spectroscopy or for exposure testing. In another embodiment, internal light/radiation source 964 can also be beneficially used for locating and aligning the geometric center of the nozzle 907. In this aspect, light emitted and passing through nozzle 907 casts a projection of the nozzle orifice onto the sample surface, allowing the nozzle's geometric center to be estimated. This aspect is particularly useful when, as shown in FIG. 9E, the NMA 935, and therefore the jet, is positioned at an angle relative to the sample and alignment of the jet-nozzle-RFL imaging/measurement beams can be difficult. Depending on the embodiment, installing multiple light/radiation sources may be beneficial, including for example, placing a light within the stage 990 for inspection of transparent samples (not shown).

In some embodiments, it should be appreciated that the manner window 927, light/radiation sources 960, 962 and 964 are integrated into RFL NMA 900 is a matter of design choice and application needs. Although these as well as other elements are depicted as being removable, in certain embodiments they may not be removable (i.e., window 927 can be permanently installed/fixed to nozzle manifold 933 or light/radiation source 964 is placed below window 927). In some applications the ability to remove light/radiation source 960 and/or 964 can be beneficial. For example, exchanging lights with ones having a different output wavelength(s) can be useful in chemical sensing applications (e.g. florescence) or photo exposure testing (e.g. photoresists).

Also shown in FIGS. 9A-F is a light diffuser or light source 965. In some embodiments, light diffuser or light source 965 is configured to provide a white background to which substrate 950 is superimposed thereon. In some further embodiments, light diffuser or light source 965 may include a diffuse light source such as a light emitting diode (LED) or red, green, blue (RGB) panel. Another way to say the same thing is, the diffuser-light source 965 is superimposed onto substrate 950. In certain embodiments, such as that shown in FIGS. 9A-F, a modular RFL NMA 900 with two separate mounts/stands, i.e., phone RFL mount/stand 985/986 and NMA mount/stand 987/988 may be used that allow variable angular positioning. In these embodiments, the smartphone-RFL can be positioned at an angle (θ) during imaging, while the NMA's nozzle axis is perpendicular to the sample and stage like depicted in FIG. 9F. When imaging at an oblique angle, light or light diffuser 965 shown attached to stage 990 can be used to improve image, video and/or measurement quality.

The exact design, construction and integration of the various RFL NMA 900 elements, including primary and secondary mirrors, window(s), light/radiation source(s), lenses, nozzle, seals, fasteners, sensors, and wires dimensions etc. require consideration of, but not limited to, the desired optical performance, volumetric flow rates, thermal isolation, and measurement dynamic range (e.g. viscosity, film thickness). One with skill in the art should therefore consider these various aspects when designing and, selecting sensors and materials to construct the RFL NMA 900 for a particular application.

It should be appreciated that, depending on the embodiment, the NMA described herein may be a separate component that attaches to a functional RFL. In some embodiments, both NMA and RFL are functional devices on their own. The embodiments with RFL NMA may be one assembled component and may not be readily separated from each other.

Certain embodiments may use a diffuse light source, such as a small LED panel, that replaces the diffuser but also provides the same function (i.e., improve lighting, image quality and contrast of the sample being analyzed and of the stagnation zone during measurements). An RGB panel with color control in other embodiments may be used to track interference fringes inside and outside the measurement location and should allow reconstructing 3D thickness maps from the images.

Each configuration of RFL NMA, as shown in and described with respect to FIGS. 1-9, can be used to construct Coaxial Thin Film Viscometers (CTFV), a preferred type of TFV instrument that can measure the interfacial-rheological-optical properties of thin fluid films and the surfaces that they coat. RFL NMAs described in these embodiments facilitate coaxial alignment between the film thickness measurement location and/or optical image with a normal impinging jet's stagnation point. Further, the RFL NMAs allow simultaneous monitoring and control of the impinging jet, and RFL and NMA components. These RFL NMA attributes may be preferred for construction of RFL-TFV instruments as they enable routine, local physical property analysis of fluids resting on solid material surfaces, simplify the thin fluid film measurement process, expand measurement protocols, improve measurement accuracy and precision, and ease theoretical analysis. CTFVs ensure coaxial alignment of the imaging and film thickness measurement location with an impinging jet's stagnation point, while simultaneously monitoring and controlling the impinging jet properties. With high precision components, such as machining and assembly, a person of ordinary skill in the art may construct the RFL NMA without positioners to align the imaging and film thickness measurement location with the nozzle and impinging jet's stagnation point (i.e., align their respective centers and/or centerlines). In this regard, the components are fixed once assembled and in principle would not require further adjustment. For purposes of explanation/clarification, it may be beneficial to include positioners within the NMA, nozzle and/or RFL so that the components can be moved relative to each other by the user. Positioners may include mounting features with precision adjustment screws and stages that give the user flexibility to make measurements when coaxially aligned or to make measurements outside the stagnation point/zone. When positioners are included, there are several positioner combinations, however it is a matter of design choice which component(s) (RFL, NMA, nozzle, etc.) the positioners are incorporated into. During practice, a person of ordinary skill in the art may fix one, say the RFL, and have XYZ translation for the other components.

It will be readily understood that the components of various embodiments of the present invention, as generally described and illustrated in the figures herein, may be arranged and designed in a wide variety of different configurations. Thus, the detailed description of the embodiments of the present invention, as represented in the attached figures, is not intended to limit the scope of the invention as claimed but is merely representative of selected embodiments of the invention.

It should be appreciated that, depending on the embodiment, the NMA described herein may be a separate component that attaches to a functional RFL. In some embodiments, both NMA and RFL are functional devices on their own. The embodiments with RFL NMA may be one assembled component and may not be readily separated from each other.

Certain embodiments may use a diffuse light source, such as a small LED panel, that replaces the diffuser but also provides the same function (i.e., improve lighting, image quality and contrast of the sample being analyzed and of the stagnation zone during measurements). An RGB panel with color control in other embodiments may be used to track interference fringes inside and outside the measurement location and should allow reconstructing 3D thickness maps from the images.

Each configuration of RFL NMA, as shown in and described with respect to FIGS. 1-9, can be used to construct Coaxial Thin Film Viscometers (CTFV), a preferred type of TFV instrument that can measure the interfacial-rheological-optical properties of thin fluid films and the surfaces that they coat. RFL NMAs described in these embodiments facilitate coaxial alignment between the film thickness measurement location and/or optical image with a normal impinging jet's stagnation point. Further, the RFL NMAs allow simultaneous monitoring and control of the impinging jet, and RFL and NMA components. These RFL NMA attributes may be preferred for construction of RFL-TFV instruments as they enable routine, local physical property analysis of fluids resting on solid material surfaces, simplify the thin fluid film measurement process, expand measurement protocols, improve measurement accuracy and precision, and ease theoretical analysis. CTFVs ensure coaxial alignment of the imaging and film thickness measurement location with an impinging jet's stagnation point, while simultaneously monitoring and controlling the impinging jet properties. With high precision components, such as machining and assembly, a person of ordinary skill in the art may construct the RFL NMA without positioners to align the imaging and film thickness measurement location with the nozzle and impinging jet's stagnation point (i.e., align their respective centers and/or centerlines). In this regard, the components are fixed once assembled and in principle would not require further adjustment. For purposes of explanation/clarification, it may be beneficial to include positioners within the NMA, nozzle and/or RFL so that the components can be moved relative to each other by the user. Positioners may include mounting features with precision adjustment screws and stages that give the user flexibility to make measurements when coaxially aligned or to make measurements outside the stagnation point/zone. When positioners are included, there are several positioner combinations, however it is a matter of design choice which component(s) (RFL, NMA, nozzle, etc.) the positioners are incorporated into. During practice, a person of ordinary skill in the art may fix one, say the RFL, and have XYZ translation for the other components.

It will be readily understood that the components of various embodiments of the present invention, as generally described and illustrated in the figures herein, may be arranged and designed in a wide variety of different configurations. Thus, the detailed description of the embodiments of the present invention, as represented in the attached figures, is not intended to limit the scope of the invention as claimed but is merely representative of selected embodiments of the invention.

The features, structures, or characteristics of the invention described throughout this specification may be combined in any suitable manner in one or more embodiments. For example, reference throughout this specification to “certain embodiments,” “some embodiments,” or similar language means that a particular feature, structure, or characteristic described in connection with the embodiment is included in at least one embodiment of the present invention. Thus, appearances of the phrases “in certain embodiments,” “in some embodiment,” “in other embodiments,” or similar language throughout this specification do not necessarily all refer to the same group of embodiments and the described features, structures, or characteristics may be combined in any suitable manner in one or more embodiments.

It should be noted that reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

One having ordinary skill in the art will readily understand that the invention as discussed above may be practiced with steps in a different order, and/or with hardware elements in configurations which are different than those which are disclosed. Therefore, although the invention has been described based upon these preferred embodiments, it would be apparent to those of skill in the art that certain modifications, variations, and alternative constructions would be apparent, while remaining within the spirit and scope of the invention. In order to determine the metes and bounds of the invention, therefore, reference should be made to the appended claims.

1. A reflective lens (RFL) nozzle manifold assembly (NMA) device (the “device”), comprising:
a reflective lens assembly including at least one reflective optical element configured to manipulate and direct electromagnetic radiation to spatial coordinates within or outside the reflective lens assembly; and
a nozzle manifold assembly coupled to the reflective lens assembly, the nozzle manifold assembly comprising
a support structure,
a second reflective optical element supported by the support structure,
a nozzle supported by the support structure, and
one or more fluid-flow features configured to receive a fluid and direct the fluid to the nozzle to form an impinging jet, wherein
the reflective lens assembly and the nozzle manifold assembly are arranged such that at least one reflected beam, imaging beam and or measurement beam is alignable with the nozzle.

2. The device of claim 1, wherein the nozzle manifold assembly comprises one or more positioners to move the nozzle manifold assembly or the nozzle relative to an axis of the at least one reflected beam, imaging beam and or measurement beam, thereby adjusting a center of an impinging jet relative to the at least one beam or measurement beam.

3. The device of claim 1, further comprising:
one or more gaskets or seals associated with the reflective lens assembly, the nozzle manifold assembly, support structure, the nozzle or any combinations thereof and configured to direct the fluid to exit through the nozzle.

4. The device of claim 1, wherein the support structure comprises one or more optically transparent elements or windows,
the one or more optically transparent elements or windows are configured to create a seal or chamber for the nozzle manifold assembly to direct supply fluid to the nozzle, and
the one or more optically transparent elements or windows are to hold reflective optical elements, to hold a removable nozzle, to include a plurality of integrated nozzles, to include a plurality of sensor-control devices or any combinations thereof.

5. The device of claim 1, wherein the one or more fluid-flow features comprising one or more tubes, channels, holes, bores, ports, or cutouts configured to supply the fluid to the nozzle or integrate one or more sensor-control devices to monitor and/or control one or more properties of the device or the impinging jet, or both.

6. The device of claim 5, wherein the one or more sensor-control devices extending through or integrated with one of the tubes, channels, holes, bores, or ports in the nozzle manifold assembly, all of which are sealed to inhibit leakage of the fluid.

7. The device of claim 1, wherein the support structure comprises at least one transparent optical element comprising a refractive lens configured to compensate for one or more optical artifacts introduced by the RFL NMA device.

8. A reflective imaging lens (RFL) nozzle manifold assembly (NMA) device (the “device”), comprising:
a reflective imaging lens assembly comprising a body, a primary reflective optical element, a support structure being a spider mount with spider vanes carrying a second reflective optical element, and a transparent optical element configurable to be positioned in a path of at least one reflected beam, imaging beam and or measurement beam; and
a nozzle manifold assembly coupled to the reflective imaging lens assembly, the nozzle manifold assembly comprising a manifold body and a nozzle configured to receive a fluid from the fluid supply and form an impinging jet, wherein
at least one reflected beam, imaging beam and or measurement beam is alignable with the nozzle manifold assembly, nozzle or both.

9. The device of claim 8, wherein the transparent optical element and a reflective imaging lens, either of which are configured to provide a seal to ensure the fluid supplied is directed to exit through or discharge from the nozzle.

10. The device of claim 8, wherein the nozzle manifold assembly comprises the one or more fluid-flow features,
the one or more fluid-flow features comprising one or more tubes, channels, holes, bores, ports, or cutouts configured to supply the fluid to the nozzle or to integrate one or more sensor-control devices to monitor or control one or more properties of the device or the impinging jet, or both.

11. The device of claim 8, wherein the transparent optical element comprises a refractive lens configured to compensate for one or more optical artifacts introduced by the device.

12. The device of claim 8, wherein the nozzle manifold assembly is removably coupled to the reflective imaging lens assembly to permit interchange of nozzle manifold assemblies having different nozzle types, number of nozzles, geometries and/or dimensions.

13. A nozzle manifold assembly (NMA), comprising:
a nozzle coupled to or integrated with a manifold body, an optically transparent window, or a NMA component;
one or more fluid-flow features formed in or coupled to the manifold body, a spider mount-vane feature, the optically transparent window or any combinations thereof and configured to receive a fluid and direct the fluid to the nozzle to form an impinging jet;
one or more sensor-integration features configured to receive one or more sensor-control devices for monitoring and or controlling one or more properties of the nozzle manifold assembly, the impinging jet or both; and
one or more mounting features configured to couple the nozzle manifold assembly to an optical system, an inspection system, a support structure or any combinations thereof.

14. The NMA of claim 13, wherein the nozzle is removably coupled to permit interchange of nozzles having different arrangements, number of nozzles, geometries and/or dimensions.

15. The NMA of claim 13, wherein the nozzle manifold assembly comprises one or more fluid-flow features,
the one or more fluid-flow features comprising one or more tubes, channels, holes, bores, ports, or cutouts configured to supply the fluid to the nozzle or to integrate one or more sensor-control devices to monitor and/or control one or more properties of the device or the impinging jet, or both.

16. The NMA of claim 13, further comprising:
at least one transparent optical element comprises a refractive lens configured to compensate for one or more optical artifacts introduced by the NMA.

17. The NMA of claim 13, further comprising:
one or more positioners to move the nozzle manifold assembly or the nozzle relative to an axis of at least one reflected beam, imaging beam and or measurement beam, thereby adjusting a center of an impinging jet relative to the at least one beam or measurement beam.

18. The NMA of claim 13, further comprising:
a light or radiation source mounted to, or internal to, the NMA, and is configured to emit one or more wavelengths of light or radiation.

19. The NMA of claim 13, further comprising:
at least one reflective lens configured to direct electromagnetic radiation to spatial coordinates within or outside a reflective lens assembly.

20. The NMA of claim 13, further comprising:
a high emissivity material for non-contact temperature sensing purposes.
